(19) United States
(12) Patent Application Publication
(10) Pub. No.: US 2025/0295768 A1
(43) Pub. Date: Sep. 25, 2025

(54) METHODS OF TREATING AUTOIMMUNE AND ALLOIMMUNE DISORDERS

(71) Applicant: Bioverativ USA Inc., Waltham, MA (US)

(72) Inventors: Graham Parry, Cambridge, MA (US); Pavel A. Nikitin, Southe San Francisco, CA (US); Sandip Panicker, South San Francisco, CA (US)

(73) Assignee: Bioverativ USA Inc., Waltham, MA (US)

(21) Appl. No.: 18/966,631

(22) Filed: Dec. 3, 2024

Related U.S. Application Data
(63) Continuation of application No. 17/469,625, filed on Sep. 8, 2021, now abandoned.
Application No. 17/469,625 is a continuation of application No. 15/739,622, filed on Dec. 22, 2017, now abandoned.
(60) Provisional application No. 62/185,362, filed on Jun. 26, 2015.

(51) Int. Cl. A61K 39/395; A61K 35/15; A61K 39/00; C07K 16/40; C07K 16/42

(52) U.S. Cl. CPC A61K 39/39566; A61K 35/15; A61K 39/395; A61K 39/39541; C07K 16/40; C07K 16/42; A61K 2039/505; C07K 2317/24; C07K 2317/567; C07K 2317/76

(57) ABSTRACT

The present disclosure provides methods of treating an alloimmune or autoimmune disorder in an individual; the methods involve administering to the individual an effective amount of an antibody specific for complement component C1s. The present disclosure provides a method of monitoring the efficacy of a subject treatment method; the method involves detecting the level of autoantibody or alloantibody in a biological sample obtained from the individual.

### CROSS-REFERENCE

This application claims the benefit of U.S. Provisional Patent Application No. 62/185,362, filed Jun. 26, 2015, which application is incorporated herein by reference in its entirety.

### INTRODUCTION

The complement system is a well-known effector mechanism of the immune response, providing not only protection against pathogens and other harmful agents but also recovery from injury. The complement pathway comprises a number of proteins that typically exist in the body in inactive form. The classical complement pathway is triggered by activation of the first component of complement, referred to as the C1 complex, which consists of C1q, C1r, and C1s proteins. Upon binding of C1 to an immune complex or other activator, the C1s component, a diisopropyl fluorophosphate (DFP)-sensitive serine protease, cleaves complement components C4 and C2 to initiate activation of the classical complement pathway. The classical complement pathway appears to play a role in many diseases and disorders, including autoimmune disorders and alloimmune disorders.

There is a need in the art for compounds that treat a complement-mediated disease or disorder.

### SUMMARY

The present disclosure provides methods of treating an alloimmune or autoimmune disorder in an individual; the methods involve administering to the individual an effective amount of an antibody specific for complement component C1s. The present disclosure provides a method of monitoring the efficacy of a subject treatment method; the method involves detecting the level of autoantibody or alloantibody in a biological sample obtained from the individual.

### BRIEF DESCRIPTION OF THE DRAWINGS

FIG. 1 depicts the amino acid sequence of Homo sapiens complement C1s protein (SEQ ID NO:158).

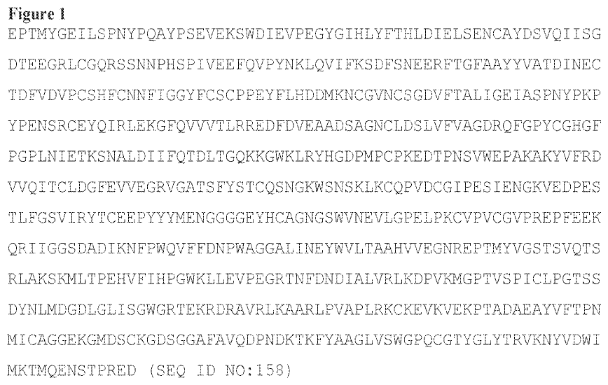

FIG. 2A-2D depict the effect of TNT003 on normal primary human B cell activation induced by B cell receptor agonists in the presence of normal human serum.

FIG. 3A-3C depict the effect of a humanized variant of TNT003 on normal primary human B cell activation and proliferation induced by a B cell receptor agonist in the presence of normal human serum.

FIG. 4A-4C depict the effect of C1s inhibitor (a humanized variant of TNT003), and C5 inhibitor antibody, on normal primary human B cell activation induced by a B cell receptor agonist in the presence of normal human serum.

FIG. 5A-5C depict the effect of various C1s inhibitor antibodies, and a C5 inhibitor antibody, on normal primary human B cell activation induced by a B cell receptor agonist in the presence of normal human serum.

### DEFINITIONS

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds. An antibody can comprise heavy- and/or light-chain constant regions of any isotype; for example, an antibody can be an IgG1, IgG2a, IgG2b, IgG3, or IgG4, and can have lambda or kappa light chains. The heavy chain constant region can be a variant with altered (e.g., increased) binding to an Fc receptor (e.g., FcRn).

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin, joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a CDR derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B11; Neuberger. M. S. et al., WO 86/01533; Neuberger. M. S. et al., European Patent No. 0,194,276 B1; Winter. U.S. Pat. No. 5,225,539; Winter, European Patent No. 0,239,400 B1; Padlan, E. A. et al., European Patent Application No. 0,519,596 A1. See also. Ladner et al., U.S. Pat. No. 4,946,778; Huston. U.S. Pat. No. 5,476,786; and Bird, R. E. et al., Science, 242: 423-426 (1988)), regarding single chain antibodies.

For example, humanized immunoglobulins can be produced using synthetic and/or recombinant nucleic acids to prepare genes (e.g., cDNA) encoding the desired humanized chain. For example, nucleic acid (e.g., DNA) sequences coding for humanized variable regions can be constructed using PCR mutagenesis methods to alter DNA sequences encoding a human or humanized chain, such as a DNA template from a previously humanized variable region (see e.g., Kamman. M., et al., Nucl. Acids Res., 17: 5404 (1989); Sato. K., et al., Cancer Research, 53: 851-856 (1993): Daugherty, B. L. et al., Nucleic Acids Res., 19(9): 2471-2476 (1991); and Lewis. A. P, and J. S. Crowe, Gene, 101: 297-302 (1991)). Using these or other suitable methods, variants can also be readily produced. For example, cloned variable regions can be mutagenized, and sequences encoding variants with the desired specificity can be selected (e.g., from a phage library; see e.g., Krebber et al., U.S. Pat. No. 5,514,548; Hoogenboom et al., WO 93/06213, published Apr. 1, 1993)).

31 “Antibody fragments” comprise a portion of an intact antibody, for example, the antigen binding or variable region of the intact antibody. Examples of antibody fragments include Fab, Fab′, F(ab′), and Fv fragments; diabodies; linear antibodies (Zapata et al., Protein Eng, 8(10): 1057-1062 (1995)): domain antibodies (dAb; Holt et al. (2003) Trends Biotechnol, 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields a F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH₁) of the heavy chain. Fab fragments differ from Fab′ fragments by the addition of a few residues at the carboxyl terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD. IgE, IgG, and IgM, and several of these classes can be further divided into subclasses (isotypes). e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404.097; WO 93/11161; and Hollinger et al. (1993) Proc. Natl. Acad. Sci. USA 90:6444-6448.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1.000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. A subject anti-C1s antibody binds specifically to an epitope within a complement C1s protein. “Specific binding” refers to binding with an affinity of at least about 10⁻⁷ M or greater. e.g., 5×10⁻⁷ M, 10⁻⁸ M, 5×10⁻⁸ M, and greater. “Non-specific binding” refers to binding with an affinity of less than about 10⁻⁷ M, e.g., binding with an affinity of 10⁻⁶ M, 10⁻⁵ M, 10⁻⁴ M, etc.

As used herein, the term “CDR” or “complementarity determining region” is intended to mean the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services. “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 1 as a comparison. The CDRs listed in Table 2 were defined in accordance with Kabat 1991.

TABLE 1
CDR Definitions

| | Kabat¹ | Chothia² | MacCallum³ |
|---|---|---|---|
| V_{H} CDR-1 | 31-35 | 16-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. As used herein, the terms “CDR-H1”, “CDR-12”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

As used herein, the term “framework” when used in reference to an antibody variable region is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

An “isolated” antibody is one that has been identified and separated and/or recovered from a component of its natural environment. Contaminant components of its natural environment are materials that would interfere with diagnostic or therapeutic uses for the antibody, and can include enzymes, hormones, and other proteinaceous or nonproteinaceous solutes. In some embodiments, the antibody will be purified (i) to greater than 90%, greater than 95%, or greater than 98%, by weight of antibody as determined by the Lowry method, for example, more than 99% by weight. (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated antibody includes the antibody in situ within recombinant cells since at least one component of the antibody's natural environment will not be present. In some instances, isolated antibody will be prepared by at least one purification step.

The terms “polypeptide,” “peptide.” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

As used herein, the terms “treatment.” “treating.” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host.” and “patient,” used interchangeably herein, refer to a mammal, including, but not limited to, murines (rats, mice), non-human primates, humans, canines, felines, ungulates (e.g., equines, bovines, ovines, porcines, caprines), etc. Also encompassed by these terms are any animal that has a complement system, such as mammals, fish, and some invertebrates. As such these terms include complement system-containing mammal, fish, and invertebrate companion animals, agricultural animals, work animals, zoo animals, and lab animals.

A “therapeutically effective amount” or “efficacious amount” refers to the amount of an anti-complement C1s antibody that, when administered to a mammal or other subject for treating a disease, is sufficient to effect such treatment for the disease. The “therapeutically effective amount” will vary depending on the anti-complement C1s antibody, the disease and its severity and the age, weight, etc., of the subject to be treated.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an anti-C1s antibody” includes a plurality of such antibodies and reference to “the autoimmune disorder” includes reference to one or more autoimmune disorders and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of treating an alloimmune or autoimmune disorder in an individual: the methods involve administering to the individual an effective amount of an antibody specific for complement component C1s in an amount and for a period of time effective to reduce the level of autoantibody or alloantibody titers. The present disclosure provides a method of monitoring the efficacy of a subject treatment method; the method involves detecting the level of autoantibody or alloantibody in a biological sample obtained from the individual.

### Treatment Methods

The present disclosure provides methods of treating an alloimmune or autoimmune disorder in an individual. The methods comprise administering to the individual an effective amount of an antibody specific for complement component C1s. The anti-C1s antibody is administered in an amount and for a period effective to reduce the level of autoantibody or alloantibody titers. Administering the anti-C1s antibody is effective to reduce the level of autoantibody or alloantibody in the individual.

### Reducing the Level of Autoimmune Antibody

In some cases, an effective amount of an anti-C1s antibody is an amount that, when administered in one or more doses and over a period of time to an individual having an autoimmune disorder, is effective to reduce the level of autoantibody in the individual by at least 10%, at least 15%, at least 20%, at least 25%, at least 30%, at least 35%, at least 40%, at least 45%, at least 50%, at least 60%, at least 70%, at least 80%, at least 90%, or more than 90%, compared to the level of autoantibody in the individual in the absence of treatment with the anti-C1s antibody, or compared to the level of autoantibody in the individual before treatment with the anti-C1s antibody.

Autoantibodies include, e.g., an anti-nuclear antibody, an anti-neutrophil antibody, an anti-ribonucleic protein antibody, an anti-single-stranded DNA antibody, an anti-La/SSA antibody, an anti-La/SS-B antibody, an anti-centromere antibody, an anti-neuronal nuclear antibody-2, an anti-double-stranded DNA antibody, an anti-Jol antibody (where the autoantigen is histidine-tRNA ligase), an anti-Smith antibody (where the autoantigen is an snRNP core protein), an anti-topoisomerase antibody, an anti-histone antibody, an anti-p62 antibody (where the autoantigen is nucleoporin 62), an anti-sp100 antibody (where the autoantigen is sp100 nuclear antigen), an anti-transglutaminase antibody, an anti-ganglioside antibody, an anti-thrombin antibody, an anti-actin antibody, an anti-neutrophil cytoplasmic antibody, an anti-signal recognition particle antibody, an anti-DNA antibody, an anti-Rho antibody, an anti-collagen antibody, an anti-1 antigen antibody, an anti-i antigen antibody, an anti-collagen XVII antibody, an anti-Rho/SSA antibody, an anti-phospholipid antibody, an anti-smooth muscle (anti-Sm) antibody, an anti-mitochondrial antibody, an anti-acetylcholine receptor antibody, an antibody to histidyl tRNA synthetase (HisRS), an anti-voltage-gated calcium channel antibody, an anti-voltage-gated potassium channel antibody, an anti-glycoprotein IIb/IIIa antibody, an anti-glycoprotein Ib/IX antibody, cold agglutinins (e.g., antibody that binds a red blood cell, such as an anti-I antigen antibody, an anti-i antigen antibody, an anti-Pr antigen antibody, etc.), an anti-aquaporin 4 antibody, an anti-muscle-specific kinase (MuSK) antibody, and the like. Autoantibodies include antibodies to autoantigens such as myelin basic protein, collagen (e.g., collagen type XI, collagen type XVII), human cartilage gp 39, chromogranin A, gp130-RAPS, proteolipid protein, fibrillarin, Rho autoantigen, I-antigen, i antigen, Pr antigen, nuclear proteins, nucleolar proteins (e.g., small nucleolar protein), thyroid stimulating factor receptor, histones, glycoprotein gp 70, ribosomal proteins, pyruvate dehydrogenase dehydrolipoamide acetyltransferase, hair follicle antigens, IgG, human tropomyosin isoform 5, mitochondrial proteins, pancreatic P-cell proteins, myelin oligodendrocyte glycoprotein, insulin, glutamic acid decarboxylase (GAD), gluten, acetylcholine receptors, aquaporin 4, muscle-specific kinase (MuSK), glycoprotein IIb/IIIa, glycoprotein Ib/IX, red blood cell antigens, platelet antigens, and the like.

Methods of determining the level of autoantibody are known in the art, and any known method can be used. Examples of suitable methods include immunological methods such as enzyme-linked immunosorbent assays (ELISA), lateral flow immunoassays (LFIA; also known as lateral flow immunochromatographic assays), diffusion immunoassays (DIA), fluoroimmunoassays (FIA), chemiluminescent immunoassays (CLIA) counting immunoassays (CIA), magnetic immunoassays (MIA), radioimmunoassays (RIA), and the like. For example, a delectably labeled autoantigen can be used in an assay to detect an autoantibody, respectively. Autoantibody present in a biological sample obtained from an individual being treated can be immobilized; and the detectably labeled autoantigen contacted with the immobilized autoantibody, forming a complex, where the presence or amount of detectable label indicates the presence or amount of autoantibody in the biological sample.

In some cases, a treatment method of the present disclosure comprises: a) administering to the individual an antibody that specifically binds complement C1s in an amount and for a period effective to reduce the level of autoantibody titers; and b) detecting a level of autoantibody in a biological sample obtained from the individual. A level of autoantibody in a biological sample obtained from the individual that is lower than a pre-treatment level can indicate efficacy of treatment. A level of autoantibody in a biological sample obtained from the individual that is not significantly lower than a pre-treatment level can indicate the need to increase the dose and/or duration of administration and/or the frequency of administration. A level of autoantibody in a biological sample obtained from the individual that is higher than a pre-treatment level can indicate the need to increase the dose and/or duration of administration and/or the frequency of administration.

In some cases, a treatment method of the present disclosure comprises: a) administering to the individual an antibody that specifically binds complement C1s in an amount and for a period effective to reduce the level of autoantibody liters; b) detecting a level of autoantibody in a biological sample obtained from the individual; and c) adjusting the dose of the anti-C1s antibody based on the detected level.

In some cases, an effective amount of an anti-C1s antibody is an amount that, when administered in one or more doses and over a period of time to an individual having an autoimmune disorder, is effective to reduce B-cell activation in the individual by at least 10%, at least 15%, at least 20%, at least 25%, at least 30%, at least 35%, at least 4(0%, at least 45%, at least 50%, at least 60%, at least 70%, or at least 80%, compared to the level of B-cell activation in the individual in the absence of treatment with the anti-C1s antibody, or compared to the level of B-cell activation in the individual before treatment with the anti-C1s antibody.

The present disclosure provides a method of reducing B-cell activation in an individual having an autoimmune disorder, the method comprising administering to the individual an effective amount of an antibody specific for complement component C1s. The anti-C1s antibody is administered in an amount and for a period effective to reduce the level of B-cell activation. In some cases, efficacy of treatment is monitored following administration of the anti-C1s antibody. In some cases, the dose of anti-C1s antibody is adjusted based on the results of the monitoring. Thus, in some cases, a method of the present disclosure comprises: a) administering to the individual an effective amount of an antibody specific for complement component C1s; and b) monitoring efficacy of said administering comprising detecting a level of B-cell activation in a biological sample obtained from the individual. In some cases, a method of the present disclosure comprises: a) administering to the individual an effective amount of an antibody specific for complement component C1s: b) monitoring efficacy of said administering comprising detecting a level of B-cell activation in a biological sample obtained from the individual; and c) adjusting the dose of the anti-C1s antibody based on the detected level of B-cell activation. The biological sample comprises B cells. For example, the biological sample can be a blood sample or other liquid or tissue sample that contains B cells. The B cells can be isolated from the biological sample. [i045]B-cell activation can be determined using any convenient method including, e.g., calcium flux. Calcium flux can be determined using a fluorescent calcium indicator. Fluorescent calcium indicators are known in the art and include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Acquorin, dextran conjugates or any other derivatives of any of these dyes, and others (see, e.g., the catalog or Internet site for Molecular Probes. Eugene, see, also, Nuccitelli, ed., Methods in Cell Biology. Volume 40: A Practical Guide to the Study of Calcium in Living Cells, Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis, Second Ed. Academic Press (1999); Calcium Signaling Protocols (Methods in Molecular Biology), 2005, D. G. Lamber, ed., Humana Press.).

B-cell activation can be determined using other convenient methods including. e.g., assessing cell surface markers of B cell activation and differentiation. Cell surface activation markers include, but are not limited to, CD23, CD25, CD27, CD30, CD38, CD69, CD80, CD86, CD135 and the like, that can be monitored using flow cytometry, immunohistochemistry, immunofluorescence, and other methods utilized in the field. Additionally, cell surface markers that are specific to naive, undifferentiated B cells can be monitored to assess the proportion of naive versus activated cells in the circulation. Markers of naive cells include, but are not limited to, IgM, CD10, and other such markers. Additionally, intracellular activation markers such as transcription factors, phosphosignaling proteins, and cytokines can also be monitored to assess the activation and proliferative status of B cells. Transcription factors that can be monitored include, but are not limited to, Oct-2, Pax-5, Blimp-1, Bcl-6, XPB-1, and the like. Phosphosignaling proteins that can be monitored include, but are not limited to, phospho-Akt, phospho-Btk, phospho-Syk, phospho-BLNK, phospho-CD20/BL-CAM, phospho-IKKγ, phospho-NFκB, phospho-mTOR and the like. Cytokines that can be monitored include, but are not limited to, IL-2, IL-4, IL-6, IFN-γ, IL-10, IL-12, TNF-α, TGF-β, and the like. Assessment of transcription factors, phosphosignaling proteins and cytokines can be assessed via flow cytometry, reverse transcription-polymerase chain reaction (RT-PCR), immunofluorescence of cells, as well as enzyme-linked immunosorbent assays (ELISAs) of cytokine levels assessed in the whole blood, plasma, or serum of patients, and other methods that are known in the field. Additionally. B cell size and granularity can be monitored via flow cytometry, microscopy, and other methods known in the field, to assess the activation status of B cells.

In some cases, an effective amount of an anti-C1s antibody is an amount that, when administered in one or more doses and over a period of time to an individual having an autoimmune disorder, is effective to reduce B-cell proliferation in the individual by at least 10%, at least 15%, at least 20%, at least 25%, at least 30%, at least 35%, at least 40%, at least 45%, at least 50%, at least 60%, at least 70%, or at least 80%, compared to the level of B-cell proliferation in the individual in the absence of treatment with the anti-C1s antibody, or compared to the level of B-cell proliferation in the individual before treatment with the anti-C1s antibody.

In some cases, an effective amount of an anti-C1s antibody is an amount that, when administered in one or more doses and over a period of time to an individual having an autoimmune disorder, is effective to reduce the number of autoreactive B cells in the individual by at least 10%, at least 15%, at least 20%, at least 25%, at least 30%, at least 35%, at least 40%, at least 45%, at least 50%, at least 60%, at least 70%, or at least 80%, compared to number of autoreactive B cells in the individual in the absence of treatment with the anti-C1s antibody, or compared to the to number of autoreactive B cells in the individual before treatment with the anti-C1s antibody.

The present disclosure provides a method of reducing B-cell proliferation in an individual having an autoimmune disorder, the method comprising administering to the individual an effective amount of an antibody specific for complement component C1s. The anti-C1s antibody is administered in an amount and for a period effective to reduce the level of B-cell proliferation. In some cases, efficacy of treatment is monitored following administration of the anti-C1s antibody. In some cases, the dose of anti-C1s antibody is adjusted based on the results of the monitoring. Thus, in some cases, a method of the present disclosure comprises: a) administering to the individual an effective amount of an antibody specific for complement component C1s; and b) monitoring efficacy of said administering comprising detecting a level of B-cell proliferation in a biological sample obtained from the individual, in some cases, a method of the present disclosure comprises: a) administering to the individual an effective amount of an antibody specific for complement component C1s; b) monitoring efficacy of said administering comprising detecting a level of B-cell proliferation in a biological sample obtained from the individual; and c) adjusting the dose of the anti-C1s antibody based on the detected level of B-cell proliferation. The biological sample comprises B cells. For example, the biological sample can be a blood sample or other liquid or tissue sample that contains B cells. The B cells can be isolated from the biological sample.

B-cell proliferation can be determined using any known assay, e.g., determining the number of CD19⁺ B cells or CD20⁺ or CD21⁺ or CD22⁺ B cells (e.g., using flow cytometry, microscopy, fluorescent microscopy, a hemocytometer, and other instruments and methods known to the field.

Autoimmune disorders that can be treated using a method of the present disclosure for treating an autoimmune disorder are autoimmune disorders mediated by autoantibodies, and include, but are not limited to. Addison's disease, age-related macular degeneration, alopecia, autoimmune hepatitis (e.g., autoimmune hepatitis associated with hepatitis B virus infection; autoimmune hepatitis associated with hepatitis C virus infection), autoimmune hemolytic anemia, autoimmune skin diseases, autoimmune thyroid disease, bullous pemphigoid, celiac disease, cold agglutinin disease, dermatomyositis, type 1 diabetes mellitus, Grave's disease, Goodpasture's syndrome. Hashímoto's disease, hypoparathyroidism, hypopituitarism, hypothyroidism, idiopathic thrombocytopenic purpura, inflammatory bowel disease (e.g., Crohn's disease; ulcerative colitis), multiple sclerosis, myasthenia gravis, myocarditis, neuromyelitis optica, pemphigus vulgaris, pemphigus foliaceus, polymyositis, psoriasis, rheumatoid arthritis, sarcoidosis, scleroderma, Sjögren's syndrome, systemic lupus erythematosus, uveitis, and Wegener's granulomatosis and poly/dermatomyositis.

Diseases that can be treated using a method of the present disclosure include, e.g., age-related autoimmune disorders, age-related macular degeneration. Alzheimer's disease, amyotrophic lateral sclerosis, anaphylaxis, argyrophilic grain dementia, arthritis (e.g., rheumatoid arthritis), asthma, atherosclerosis, atypical hemolytic uremic syndrome, autoimmune diseases, autoimmune hemolytic anemia. Barraquer-Simons syndrome, Behçet's disease. British type amyloid angiopathy, bullous pemphigoid. Buerger's disease. C1q nephropathy, cancer, catastrophic antiphospholipid syndrome, cerebral amyloid angiopathy, cold agglutinin disease, corticobasal degeneration. Creutzfeldt-Jakob disease, Crohn's disease, cryoglobulinemic vasculitis, dementia pugilistica, dementia with Lewy Bodies (DLB), diffuse neurofibrillary tangles with calcification, Discoid lupus erythematosus. Down's syndrome, focal segmental glomerulosclerosis, formal thought disorder, frontotemporal dementia (FTD), frontotemporal dementia with parkinsonism linked to chromosome 17, frontotemporal lobar degeneration, Gerstmann-Straussler-Scheinker disease. Guillain-Barré syndrome, Hallervorden-Spatz disease, hemolytic-uremic syndrome, hereditary angioedema, hypophosphastasis, idiopathic pneumonia syndrome, immune complex diseases, inclusion body myositis, infectious disease (e.g., disease caused by bacterial (e.g., Neisseria meningitidis or Streptococcus) viral (e.g., human immunodeficiency virus (HIV)), or other infectious agents), inflammatory disease, ischemia/reperfusion injury, mild cognitive impairment, immunothrombocytopenic purpura (ITP), molybdenum cofactor deficiency (MoCD) type A, membranoproliferative glomerulonephritis (MPGN) 1, membranoproliferative glomerulonephritis (MPGN) II (dense deposit disease), membranous nephritis, multi-infarct dementia, lupus (e.g., systemic lupus erythematosus (SLE)), glomerulonephritis, Kawasaki disease, multifocal motor neuropathy, multiple sclerosis, multiple system atrophy, myasthenia gravis, myocardial infarction, myotonic dystrophy, neuromyelitis optica, Niemann-Pick disease type C, non-Guamanian motor neuron disease with neurofibrillary tangles, Parkinson's disease, Parkinson's disease with dementia, paroxysmal nocturnal hemoglobinuria. Pemphigus vulgaris, Pick's disease, postencephalitic parkinsonism, polymyositis, prion protein cerebral amyloid angiopathy, progressive subcortical gliosis, progressive supranuclear palsy, psoriasis, sepsis, Shiga-toxin E coli (STEC)-HuS, spinal muscular atrophy, stroke, subacute sclerosing panencephalitis, Tangle only dementia, transplant rejection, vasculitis (e.g., ANCA associated vasculitis). Wegner's granulomatosis, sickle cell disease, cryoglobulinemia, mixed cryoglobulinemia, essential mixed cryoglobulinemia. Type II mixed cryoglobulinemia, Type III mixed cryoglobulinemia, nephritis, drug-induced thrombocytopenia, lupus nephritis, bullous pemphigoid, Epidermolysis bullosa acquisita, delayed hemolytic transfusion reaction, hypocomplementemic urticarial vasculitis syndrome, pseudophakic bullous keratopathy, and platelet refractoriness.

### Reducing the Level of Alloimmune Antibody

In some cases, an effective amount of an anti-C1s antibody is an amount that, when administered in one or more doses and over a period of time to an individual in need thereof (e.g., a transplant graft or organ recipient), is effective to reduce the level of alloantibody in the individual by at least 10%, at least 15%, at least 20%, at least 25%, at least 3(0%, at least 35%, at least 40%, at least 45%, at least 50%, at least 60%, at least 70%, at least 80%, at least 90%, or more than 90% compared to the level of alloantibody in the individual in the absence of treatment with the anti-C1s antibody, or compared to the level of alloantibody in the individual before treatment with the anti-C1s antibody.

A method of the present disclosure provides for a reduction in the level of alloantibodies in an individual. Alloantibodies include antibodies to human leukocyte antigen (HLA) present on a donor tissue or organ. Alloantibodies include antibodies to any epitope present on a donor tissue, donor organ, or donor cell (e.g., red blood cell; platelet; endothelial cell; etc.).

Methods of determining the level of alloantibody are known in the art, and any known method can be used. Examples of suitable methods include immunological methods such as ELISA, LFIA, DIA, FIA, CLIA, CIA, MIA, RIA, and the like. For example, a detectably labeled alloantigen can be used in an assay to detect an alloantibody, respectively. Alloantibody present in a biological sample obtained from an individual being treated can be immobilized; and the detectably labeled alloantigen contacted with the immobilized alloantibody, forming a complex, where the presence or amount of detectable label indicates the presence or amount of alloantibody in the biological sample.

In some cases, a treatment method of the present disclosure comprises: a) administering to the individual an antibody that specifically binds complement C1s in an amount and for a period effective to reduce the level of alloantibody titers; and b) detecting a level of alloantibody in a biological sample obtained from the individual. A level of alloantibody in a biological sample obtained from the individual that is lower than a pre-treatment level can indicate efficacy of treatment. A level of alloantibody in a biological sample obtained from the individual that is not significantly lower than a pre-treatment level can indicate the need to increase the dose and/or duration of administration and/or the frequency of administration. A level of alloantibody in a biological sample obtained from the individual that is higher than a pre-treatment level can indicate the need to increase the dose and/or duration of administration and/or the frequency of administration.

In some cases, a treatment method of the present disclosure comprises: a) administering to the individual an antibody that specifically binds complement C1s in an amount and for a period effective to reduce the level of alloantibody titers; b) detecting a level of alloantibody in a biological sample obtained from the individual; and c) adjusting the dose of the anti-C1s antibody based on the detected level.

In some cases, an effective amount of an anti-C1s antibody is an amount that, when administered in one or more doses and over a period of time to an individual having an alloimmune disorder, is effective to reduce B-cell activation in the individual by at least 10%, at least 15%, at least 20%, at least 25%, at least 30%, at least 35%, at least 40%, at least 45%, at least 50%, at least 60%, at least 70%, or at least 80%, compared to the level of B-cell activation in the individual in the absence of treatment with the anti-C1s antibody, or compared to the level of B-cell activation in the individual before treatment with the anti-C1s antibody.

The present disclosure provides a method of reducing B-cell activation in an individual having an alloimmune disorder, the method comprising administering to the individual an effective amount of an antibody specific for complement component C1s. The anti-C1s antibody is administered in an amount and for a period effective to reduce the level of B-cell activation. In some cases, efficacy of treatment is monitored following administration of the anti-C1s antibody. In some cases, the dose of anti-C1s antibody is adjusted based on the results of the monitoring. Thus, in some cases, a method of the present disclosure comprises: a) administering to the individual an effective amount of an antibody specific for complement component C1s; and b) monitoring efficacy of said administering comprising detecting a level of B-cell activation in a biological sample obtained from the individual. In some cases, a method of the present disclosure comprises: a) administering to the individual an effective amount of an antibody specific for complement component C1s; b) monitoring efficacy of said administering comprising detecting a level of B-cell activation in a biological sample obtained from the individual; and c) adjusting the dose of the anti-C1s antibody based on the detected level of B-cell activation. The biological sample comprises B cells. For example, the biological sample can be a blood sample or other liquid or tissue sample that contains B cells. The B cells can be isolated from the biological sample.

B-cell activation can be determined using any convenient method including, e.g., calcium flux. Calcium flux can be determined using a fluorescent calcium indicator. Fluorescent calcium indicators are known in the art and include, but are not limited to, fura-2, bis-fura 2, indo-1, Quin-2, Quin-2 AM, Benzothiaza-1, Benzothiaza-2, indo-5F, Fura-F, BTC, Mag-Fura-2, Mag-Fura-5, Mag-Indo-1, fluo-3, rhod-2, fura-4F, fura-SE, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-SN, Fluo-5F, Fluo-4FF, Mag-Fluo-4, Aequorin, dextran conjugates or any other derivatives of any of these dyes, and others (see, e.g., the catalog or Internet site for Molecular Probes, Eugene, see, also. Nuccitelli, ed., Methods in Cell Biology, Volume 40: A Practical Guide to the Study of Calcium in Living Cells. Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis. Second Ed. Academic Press (1999); Calcium Signaling Protocols (Methods in Molecular Biology), 2005. D. G. Lamber, ed., Humana Press.).

B-cell activation can be determined using other convenient methods including. e.g., assessing cell surface markers of B cell activation and differentiation. Cell surface activation markers include, but are not limited to, CD23, CD25, CD27, CD30, CD38. CD69, CD80, CD86, CD135 and the like, that can be monitored using flow cytometry, immunohistochemistry, immunofluorescence, and other methods utilized in the field. Additionally, cell surface markers that are specific to naïve, undifferentiated B cells can be monitored to assess the proportion of naïve versus activated cells in the circulation. Markers of naïve cells include, but are not limited to. IgM, CD10, and other such markers. Additionally, intracellular activation markers such as transcription factors, phosphosignaling proteins, and cytokines can also be monitored to assess the activation and proliferative status of B cells. Transcription factors that can be monitored include, but are not limited to, Oct-2, Pax-5, Blimp-1, Bcl-6, XPB-1, and the like. Phosphosignaling proteins that can be monitored include, but are not limited to, phospho-Akt, phospho-Btk, phospho-Syk, phospho-BLNK, phospho-CD20/BL-CAM, phospho-IKKγ, phospho-NFκB, phospho-mTOR and the like. Cytokines that can be monitored include, but am not limited to, I1-2, IL-4, IL-6, IFN-γ, IL-10, IL-12, TNF-α, TGF-β, and the like. Assessment of transcription factors, phosphosignaling proteins and cytokines can be assessed via flow cytometry. RT-PCR, immunofluorescence of cells, as well as ELISAs of cytokine levels assessed in the whole blood, plasma, or serum of patients, and other methods that are known in the field. Additionally, B cell size and granularity can be monitored via flow cytometry, microscopy, and other methods known in the field, to assess the activation status of B cells.

In some cases, an effective amount of an anti-C1s antibody is an amount that, when administered in one or more doses and over a period of time to an individual having an alloimmune disorder, is effective to reduce B-cell proliferation in the individual by at least 10%, at least 15%, at least 20%, at least 25%, at least 30%, at least 35%, at least 40%, at least 45%, at least 50%, at least 60%, at least 70%, or at least 80%, compared to the level of B-cell proliferation in the individual in the absence of treatment with the anti-C1s antibody, or compared to the level of B-cell proliferation in the individual before treatment with the anti-C1s antibody.

In some cases, an effective amount of an anti-C1s antibody is an amount that, when administered in one or more doses and over a period of time to an individual having an alloimmune disorder, is effective to reduce the number of alloreactive B cells in the individual by at least 10%, at least 15%, at least 20%, at least 25%, at least 30%, at least 35%, at least 40%, at least 45%, at least 50%, at least 60%, at least 70%, or at least 80%, compared to number of alloreactive B cells in the individual in the absence of treatment with the anti-C1s antibody, or compared to the to number of alloreactive B cells in the individual before treatment with the anti-C1s antibody.

The present disclosure provides a method of reducing B-cell proliferation in an individual having an alloimmune disorder, the method comprising administering to the individual an effective amount of an antibody specific for complement component C1s. The anti-C1s antibody is administered in an amount and for a period effective to reduce the level of B-cell proliferation.

B-cell proliferation can be determined using any known assay, e.g., determining the number of CD19⁺ B cells or CD20⁺ or CD21⁺ or CD22⁺ B cells (e.g., using flow cytometry, microscopy, fluorescent microscopy, a hemocytometer, and other instruments and methods known to the field).

Alloimmune disorders that can be treated using a method of the present disclosure for treating an alloimmune disorder include antibody-mediated rejection of an allograft organ, tissue, or cell. Allograft organs, tissues, and cells include, but are not limited to, a kidney, a liver, a pancreas, a heart, a lung, skin, blood tissue (including whole blood; red blood cells; white blood cells; cord blood; and the like, where the blood tissue may comprise an isolated population of blood cells (huffy coat; red blood cells; platelets; lymphocytes; T cells; B cells; or some other population), or where the blood tissue comprises a mixed population of cells), small intestine, an endothelial tissue, a vascular tissue (e.g., a blood vessel), an eye, a stomach, a thymus, bone, bone marrow, cornea, a heart valve, an islet of Langerhans, or a tendon. As used herein, “organ” encompasses a whole organ or a part of an organ. As used herein. “tissue” encompasses a whole tissue or part of a tissue.

In some cases, a method of the present disclosure for treating an alloimmune disorder comprises administering an effective amount of an anti-C1s antibody to an individual who has received a donor organ or tissue (e.g., an organ or tissue recipient). In some cases, a method of the present disclosure for treating an alloimmune disorder comprises administering an effective amount of an anti-C1s antibody to an individual who has received a donor organ or tissue (e.g., an organ or tissue recipient), where the individual exhibits symptoms of antibody-mediated rejection (AMR). In some cases, a method of the present disclosure for treating an alloimmune disorder comprises administering an effective amount of an anti-C1s antibody to an individual who has received a donor organ or tissue (e.g., an organ or tissue recipient), where the individual has been diagnosed as having AMR. Thus, e.g., in some cases, the present disclosure provides a method of treating AMR, comprising administering to an individual who has been diagnosed as having AMR an effective amount of an anti-C1s antibody. In some cases, a method of the present disclosure provides for reducing B-cell proliferation and/or B-cell activation in an individual having AMR.

In some cases, a method of the present disclosure for treating an alloimmune disorder comprises administering an effective amount of an anti-C1s antibody to an individual who is to receive (e.g., who is scheduled to receive; who is on a wait list to receive; etc.) a donor organ, donor tissue, or donor cell (or donor cell population) (e.g., a prospective organ or tissue recipient; a prospective transfusion recipient; a prospective bone marrow transplant recipient; etc.). In some cases, a method of the present disclosure for treating an alloimmune disorder comprises administering an effective amount of an anti-C1s antibody to an individual who is to receive (e.g., who is scheduled to receive; who is on a wait list to receive; etc.) a donor organ, donor tissue, or donor cell (or donor cell population) (e.g., a prospective organ or tissue recipient; a prospective bone marrow transplant recipient; etc.), where the treatment with the anti-C1s antibody starts before the individual has received the donor organ, donor tissue, or donor cell or cell population, and where the treatment continues after the individual has received the donor organ, donor tissue, or donor cell or cell population.

In some cases, in carrying out a method of the present disclosure for treating an alloimmune disorder, an anti-C1s antibody is administered to a prospective organ or tissue recipient from 1 hour to 7 days (e.g., from 1 hour to 4 hours, from 4 hours to 8 hours, from 8 hours to 12 hours, from 12 hours to 16 hours, from 16 hours to 24 hours, from 1 day to 2 days, from 2 days to 3 days, from 3 days to 4 days, from 4 days to 5 days, from 5 days to 6 days, or from 6 days to 7 days) before receiving the organ or tissue.

### Dosages; Frequency of Administration; Duration of Administration

A suitable dosage of an anti-C1s antibody can be determined by an attending physician or other qualified medical personnel, based on various clinical factors. As is well known in the medical arts, dosages for any one patient depend upon many factors, including the patient's size, body surface area, age, the particular compound to be administered, sex of the patient, time, and route of administration, general health, and other drugs being administered concurrently. An anti-C1s antibody can be administered in amounts between 1 ng/kg body weight and 100 mg/kg body weight per dose, e.g. from 1 ng/kg body weight to 50 ng/kg body weight, from 50 ng/kg body weight to 0.1 mg/kg body weight, from 0.1 mg/kg body weight 1 mg/kg body weight, from 1 mg/kg body weight to 5 mg/kg body weight, from 5 mg/kg body weight to 10 mg/kg body weight, from 0.5 mg/kg body weight to 5 mg/kg body weight, from 10 mg/kg body weight to 20 mg/kg body weight, from 20 mg/kg body weight to 50 mg/kg body weight, or from 50 mg/kg body weight to 100 mg/kg body weight, or more than 100 mg/kg body weight; however, doses below or above this exemplary range are envisioned, especially considering the aforementioned factors. If the regimen is a continuous infusion, it can also be in the range of 1 μg to 10 mg per kilogram of body weight per minute.

In some cases, a dose of an anti-C1s antibody is in the range of 0.001 g to 1000 pg; however, doses below or above this exemplary range are envisioned, especially considering the aforementioned factors. In some cases, the dosage can range, e.g., from about 0.0001 to 100 mg/kg, or from about 0.01 to 5 mg/kg (e.g., 0.02 mg/kg, 0.25 mg/kg, 0.5 mg/kg, 0.75 mg/kg, 1 mg/kg, 2 mg/kg, etc.) body weight. For example dosages can be 1 mg/kg body weight or 10 mg/kg body weight or within the range of 1-10 mg/kg, or at least 1 mg/kg. Doses intermediate in the above ranges are also intended to be within the scope of the invention.

In some embodiments, an anti-C1s antibody is administered in an amount that provides for a peak serum concentration of from about 1 μg/ml to about 1 mg/ml, e.g., from about 1 μg/ml to about 2.5 μg/ml, from about 2.5 μg/ml to about 5 μg/ml, from about 5 μg/ml to about 7.5 μg/ml, from about 7.5 μg/ml to about 10 μg/ml, from about 10 μg/ml to about 25 μg/ml, from about 25 μg/ml to about 50 μg/ml, from about 50 μg/ml to about 100 μg/nil, from about 100 μg/ml to about 250 μg/ml, from about 250 μg/ml to about 500 g/ml, from about 500 μg/ml to about 750 μg/ml, or from about 750 μg/ml to about 1000 μg/ml. In some embodiments, an anti-C1s antibody is administered in an amount that provides for a peak serum concentration of greater than 1 mg/ml, e.g., from about 1 mg/ml to about 2 mg/ml, from about 2 mg/ml to about 5 mg/ml, or from about 5 mg/ml to about 10 mg/ml.

An anti-C1s antibody can be administered at any of a variety of frequencies. In some cases, multiple doses of an anti-C1s antibody are administered. The frequency of administration of an anti-C1s antibody can vary depending on any of a variety of factors, e.g., severity of the symptoms, etc. For example, in some cases, an anti-C1s antibody is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

In some cases, an anti-C1s antibody is administered over a period of time of 6 months or longer. In some cases, an anti-C1s antibody is administered over a period of time of from 6 months to 1 year, from 1 year to 2 years, from 2 years to 5 years, or more than 5 years.

In some cases, an anti-C1s antibody is administered over a period of time of less than 6 months. In some cases, an anti-C1s antibody is administered over a period of time of 5.5 months or less. In some cases, an anti-C1s antibody is administered over a period of time of 5 months or less. In some cases, an anti-C1s antibody is administered over a period of time of 4.5 months or less, in some cases, an anti-C1s antibody is administered over a period of time of 4 months or less. In some cases, an anti-C1s antibody is administered over a period of time of 3.5 months or less. In some cases, an anti-C1s antibody is administered over a period of time of 3 months or less. In some cases, an anti-C1s antibody is administered over a period of time of 2.5 months or less. In some cases, an anti-C1s antibody is administered over a period of time of 2 months or less, in some cases, an anti-C1s antibody is administered over a period of time of 1 month or less. In some cases, an anti-C1s antibody is administered over a period of time of 3 weeks. In some cases, an anti-C1s antibody is administered over a period of time of 2 weeks. In some cases, an anti-C1s antibody is administered over a period of time of 1 week.

An anti-C1s antibody can be administered via any of a variety of routes of administration. Conventional and pharmaceutically acceptable routes of administration include intranasal, intramuscular, intratracheal, intrathecal, intracranial, subcutaneous, intradermal, topical, intravenous, intraperitoneal, intraarterial (e.g., via the carotid artery), spinal or brain delivery, rectal, nasal, oral, and other enteral and parenteral routes of administration. Routes of administration can be combined, if desired, or adjusted depending upon the antibody and/or the desired effect. In some cases, an anti-C1s antibody is administered subcutaneously. In some cases, an anti-C1s antibody is administered intravenously. In some cases, an anti-C1s antibody is administered intramuscularly.

### Anti-C1s Antibodies

Any of a variety of anti-C1s antibodies can be used in a method of the present disclosure of treating an alloimmune disorder or an autoimmune disorder, or in a method of reducing B-cell proliferation and/or B-cell activation. In some cases, the anti-C1s antibody is humanized. In some cases, the anti-C1s antibody comprises a humanized VH framework region. In some cases, the anti-C1s antibody comprises a humanized VL framework region. In some cases, the anti-C1s antibody comprises a humanized VH framework region and a humanized VL framework region.

As one example of a suitable anti-C1s antibody, in some cases, the anti-C1s antibody comprises VH CDRs present in the following VH amino acid sequence:

(SEQ ID NO: 100)
EVQLQQSGAELVRPGASVKLSCTASGFNIKDDYIHWVKQRPEQGLEWIGR
IDPADDHTKYAPKFQDKATMTADTSSNTACLQLNSLTSEDTAVYYCAIYG
SGWAWFPYWGQGTLVSVSA.

As one example of a suitable anti-C1s antibody, in some cases, the anti-C1s antibody comprises VL CDRs present in the following VL amino acid sequence:

(SEQ ID NO: 101)
DIVLTQSTDYLAVSLGQRATISCKASQSVDYDGDSYMNWYQQKPGQPPKL
LIYAASNLESGIPARFSGSGSGTDFTLNIHPVEEEDAATYYCQQSNEDPW
TFGGGTKLEIK.

As one example of a suitable anti-C1s antibody, in some cases, the anti-C1s antibody comprises the following VH CDRs:

(SEQ ID NO: 1)
1) CDR-H1: GFNIKDDYIHWV;
(SEQ ID NO: 2)
2) CDR-H2: IDPADDHTKY;
and
(SEQ ID NO: 3)
3) CDR-H3: AIYGSGWAWFPY.

As one example of a suitable anti-C1s antibody, in some cases, the anti-C1s antibody comprises the following VL CDRs:

(SEQ ID NO: 4)
1) CDR-L1: QSVDYDGDSYMN;
(SEQ ID NO: 5)
2) CDR-L2: AASNLESGIP;
and
(SEQ ID NO: 6)
3) CDR L3: QQSNEDPWT.

As one example of a suitable anti-C1s antibody, in some cases, the anti-C1s antibody comprises the following VH CDRs and V L CDRs:

(SEQ ID NO: 1)
1) CDR-H1: GFNIKDDYIHWV;
(SEQ ID NO: 2)
2) CDR-H2: IDPADDHTKY;
(SEQ ID NO: 3)
3) CDR-H3: AIYGSGWAWFPY;
(SEQ ID NO: 4)
4) CDR-L1: QSVDYDGDSYMN;
(SEQ ID NO: 5)
5) CDR-L2: AASNLESGIP;
and
(SEQ ID NO: 6)
6) CDR-13: QQSNEDPWT.

As one example of a suitable anti-C1s antibody, in some cases, the anti-C1s antibody comprises VH CDRs present in the following VH amino acid sequence:

(SEQ ID NO: 7)
EVKLQQSGAELVRPGASVKLSCTASGFNIKDDYIHWVKQRPEQGLEWIG
RIDPADGHTKYAPKFQVKATITADTSSNTAYLQLSSLTSEDTAVYYCAR
YGYGREVFDYWGQGTTLTVSS.

As one example of a suitable anti-C1s antibody, in some cases, the anti-C1s antibody comprises VL CDRs present in the following VL amino acid sequence:

(SEQ ID NO: 8)
DIVLTQSTDYLAVSLGQRATISCKASQSVDYDGDSYMNWYQQKPGQPP
KLLIYAASNLESGIPARFSGSGSGTDFTLNIHPVEEEDAATYYCQQSN
EDPWTFGGGTKLEIK.

As one example of a suitable anti-C1s antibody, in some cases, the anti-C1s antibody comprises the following VH CDRs:

(SEQ ID NO: 9)
1) CDR-H1: GFNIKDDYIHWV;
(SEQ ID NO: 10)
2) CDR-H2: IDPADGHTKY;
and
(SEQ ID NO: 11)
3) CDR-H3: ARYGYGREVFDY.

As one example of a suitable anti-C1s antibody, in some cases, the anti-C1s antibody comprises the following VL CDRs:

(SEQ ID NO: 12)
1) CDR-L1: QSVDYDGDSYMN;
(SEQ ID NO: 13)
2) CDR-L2: DASNLESGIP;
and
(SEQ ID NO: 14)
3) CDR-L3: QQSNEDPWT.

As one example of a suitable anti-C1s antibody, in some cases, the anti-C1s antibody comprises the following VH CDRs and VL CDRs:

(SEQ ID NO: 9)
1) CDR-H1: GFNIKDDYIHWV;
(SEQ ID NO: 10)
2) CDR-H2: IDPADGHTKY;
(SEQ ID NO: 11)
3) CDR-H3: ARYGYGREVFDY;
(SEQ ID NO: 12)
4) CDR-L1: QSVDYDGDSYMN;
(SEQ ID NO: 13)
5) CDR-L2: DASNLESGIP;
and
(SEQ ID NO: 14)
6) CDR-L3: QQSNEDPWT.

As one example of a suitable anti-C1s antibody, in some cases, the anti-C1s antibody comprises VH CDRs present in the following VH amino acid sequence:

(SEQ ID NO: 102)
QVQLQQPGAELVRPGASVKLSCKVSGYTFTRYWMHWVKQRPGQGLEWIGE
INPSNSDTDYNEEFKSKATLTVDKSSSTAYMHLSSLTSEDSAVYYCTIDD
SAYGWFAYWGQGTLVTVSA.

As one example of a suitable anti-C1s antibody, in some cases, the anti-C1s antibody comprises VL CDRs present in the following V L amino acid sequence:

(SEQ ID NO: 103)
DIVMTQSPAIMSASPGERVTMTCSASSSISYMHWYHQKPGTSPKRWIYDT
SKLASGVPARFSGSGSGTSYSLTISSMEAEDAATYYCHQRSSFPTFGAGT
KLELK.

As one example of a suitable anti-C1s antibody, in some cases, the anti-C1s antibody comprises the following VH CDRs:

1) CDR-H1:
(SEQ ID NO: 15)
GYTFTRYWMHWV;
2) CDR-H2:
(SEQ ID NO: 16)
INPSNSDTDY;
and
3) CDR-H3:
(SEQ ID NO: 17)
TIDDSAYGWFAY.

As one example of a suitable anti-C1s antibody, in some cases, the anti-C1s antibody comprises the following VL CDRs:

1) CDR-L1:
(SEQ ID NO: 18)
SSISYMHWYHQK;
2) CDR-L2:
(SEQ ID NO: 19)
DTSKLASGVP:
and
3) CDR-L3:
(SEQ ID NO: 20)
HQRSSFPT.

As one example of a suitable anti-C1s antibody, in some cases, the anti-C1s antibody comprises the following VH CDRs and VL CDRs:

1) CDR-H1:
(SEQ ID NO: 15
GYTFTRYWMHWV;
2) CDR-H2:
(SEQ ID NO: 16)
INPSNSDTDY;
3) CDR-H3:
(SEQ ID NO: 17)
TIDDSAYGWFAY.
4) CDR-L1:
(SEQ ID NO: 18)
SSISYMHWYHQK;
5) CDR-L2:
(SEQ ID NO: 19)
DTSKLASGVP;
and
6) CDR-L3:
(SEQ ID NO: 20)
HQRSSFPT.

As one example of a suitable anti-C1s antibody, in some cases, the anti-C1s antibody comprises VH CDRs present in the following VH amino acid sequence:

(SEQ ID NO: 104)
QVQLQQPGAELVRPGASVKLSCKVSGYTFTRYWMHWVKQRPGQGLEWIGE
INPSNSDTDYNEEFKSKATLTVDKSSSTAYMHLSSLTSEDSAVYNCTIDD
SVYGWFAYWGQGTLVTVSA.

As one example of a suitable anti-C1s antibody, in some cases, the anti-C1s antibody comprises VL CDRs present in the following VL amino acid sequence:

(SEQ ID NO: 105)
DIVITQSPAIMSASPGERVTMTCSASSSISYMHWYHQKPGTSPKRWIYDT
SKLASGVPARFSGSGSGTSYSLTISSMEAEDAATYYCHQRSSEPTFGAGT
KLELK.

As one example of a suitable anti-C1s antibody, in some cases, the anti-C1s antibody comprises the following VH CDRs:

1) CDR-H1:
(SEQ ID NO: 21)
GYTFTRYWMHWV;
2) CDR-H2:
(SEQ ID NO: 22)
INPSNSDTD;
and
3) CDR-H3:
(SEQ ID NO: 23)
TIDDSVYGWFAY.

As one example of a suitable anti-C1s antibody, in some cases, the anti-C1s antibody comprises the following VL CDRs:

1) CDR-L1:
(SEQ ID NO: 24)
SSISYMHWYHQK;
2) CDR-L2:
(SEQ ID NO: 25)
DTSKLASGVP;
and
3) CDR-L3:
(SEQ ID NO: 26)
HQRSSFPT.

As one example of a suitable anti-C1s antibody, in some cases, the anti-C1s antibody comprises the following VH CDRs and VL CDRs:

1) CDR-H1
(SEQ ID NO: 21)
GYTFTRYWMHWV;
2) CDR-H2:
(SEQ ID NO: 22)
INPSNSDTDY;
3) CDR-H3:
(SEQ ID NO: 23)
TIDDSVYGWFAY;
4) CDR-L1:
(SEQ ID NO: 24)
SSISYMHWYHQK;
5) CDR-L2:
(SEQ ID NO: 25)
DTSKLASGVP;
and
6) CDR-L3:
(SEQ ID NO: 26)
HQRSSFPT.

As one example of a suitable anti-C19 antibody, in some cases, the anti-C1s antibody comprise, VH CDRs present in the following VH amino acid sequence:

(SEQ ID NO: 106)
QVQLQQSGAELVRPGASVKLSCTASGFNIKDDYIHWVKQRPEQGLEWIGR
IDPADDHTKYAPKFQDKATMTADTSSNTACLQLNSLTSEDTAVYYCAIYG
SGWAWFPYWGQGTLVSVSAAKTTAPSVYPLAPVCGDTTGSSVTLGCLVK.

As one example of a suitable anti-C1s antibody, in some cases, the anti-C1s antibody comprises VL CDRs present in the following VL amino acid sequence:

(SEQ ID NO: 107)
DIVMTQSPDYLAVSLGQRAPISCKASQSVDYDGDSYMNWYQQKPGQPPKL
LIYAASNLEFGIPTRFSGSGFGTDFPLNIHPVEEEDAATYYCQQSNEDPW
TFGGGPKLEIK.

As one example; of a suitable anti-C1s antibody, in some cases, the anti-C1s antibody comprises the following VH CDRs:

1) CDR-H1:
(SEQ ID NO: 27)
GFNIKDDYIHWV;
2) CDR-H2:
(SEQ ID NO: 28)
IDPADDHTKY;
and
3) CDR-H2:
(SEQ ID NO: 29)
AIYGSGWAWFPY.

As one example of a suitable anti-C1s antibody, in some cases, the anti-C1s antibody comprises the following VL CDRs:

1) CDR-L1:
(SEQ ID NO: 30)
QSVDYDGDSYMN;
2) CDR-L2:
(SEQ ID NO: 31)
AASNLEFGIP;
and
3) CDR-L3:
(SEQ ID NO: 32)
QQSNEDPWT.

As one example of a suitable anti-C1s antibody, in some cases, the anti-C1s antibody comprises the following VH CDRs and VL CDRs:

1) CDR-H1:
(SEQ ID NO: 27)
GFNIKDDYIHWV;
2) CDR-H2:
(SEQ ID NO: 28)
IDPADDHTKY;
3) CDR-H2:
(SEQ ID NO: 29)
AIYGSGWAWFPY;
4) CDR-L1:
(SEQ ID NO: 30)
QSVDYDGDSYMN;
5) CDR-L2:
(SEQ ID NO: 31)
AASNLEFGIP;
and
6) CDR-L3:
(SEQ ID NO: 32)
QQSNEDPWT.

As one example of a suitable anti-C1s antibody, in some cases, the anti-C1s antibody comprises VH CDRs present in the following VH amino acid sequence:

(SEQ ID NO: 108)
EVKLQQSGAELVRPGASVKLSCTASGFNIKDDYTHWVKQRPEQGLEWIGR
IDPADGHTKYAPKFQVKATITADTSSNTAYLQLSSLTSEDTAVYYCARYG
YGREVFDYWGQGTTLTVSS.

As one example of a suitable anti-C1s antibody, in some cases, the anti-C1s antibody comprises VL CDRs present in the following VL amino acid sequence:

(SEQ ID NO: 109)
DIVLTQFPTFLAVFLGQRAPISCKASQSVDYDGDSYMNWFQQKTGQPPKI
LIYDASNLEFGIPTRFSGSGFGTDFPLNIHPVEEEDAAIYFCQQSNEDPW
TFGGGPKLEIK.

As one example of a suitable anti-C1s antibody, in some cases, the anti-C1s antibody comprises the following VH CDRs:

1) CDR-H1:
(SEQ ID NO: 33)
GFNIKDDYIHWV;
2) CDR-H2:
(SEQ ID NO: 34)
IDPADGHTKY;
and
3) CDR-H3:
(SEQ ID NO: 35)
ARYGYGREVFDY.

As one example of a suitable anti-C1s antibody, in some cases, the anti-C1s antibody comprises the following VL CDRs:

1) CDR-L1:
(SEQ ID NO: 36)
QSVDYDGDSYMN;
2) CDR-L2:
(SEQ ID NO: 37)
DASNLEFGIP;
and
3) CDR-L3:
(SEQ ID NO: 38)
QQSNEDPWT.

As one example of a suitable anti-C1s antibody, in some cases, the anti-C1s antibody comprises the following VH CDRs and VL CDRs:

1) CDR-H1:
(SEQ ID NO: 33)
GFNIKDDYIHWV;
2) CDR-H2:
(SEQ ID NO: 34)
IDPADGHTKY;
3) CDR-H3:
(SEQ ID NO: 35)
ARYGYGREVFDY;
4) CDR-L1:
(SEQ ID NO: 36)
QSVDYDGDSYMN;
5) CDR-L2:
(SEQ ID NO: 37)
DASNLEFGIP;
and
6) CDR-L3:
(SEQ ID NO: 38)
QQSNEDPWT.

As one example of a suitable anti-C1s antibody, in some cases, the anti-C1s antibody comprises VH CDRs present in the following VH amino acid sequence:

(SEQ ID NO: 110)
EVKLEQSGAELVRPGASVKLSCTASGFNIKDDYIHWVKQRPEQGLEWIGR
IDPADDHTKYAPKFQDKATMTADTSSNTACLQLNSLTSEDTAVYYCAIYG
SGWAWFPYWGQGTLVSVSA.

As one example of a suitable anti-C1s antibody, in some cases, the anti-C1s antibody comprises VL CDRs present in the following VL amino acid sequence:

(SEQ ID NO: 111)
EFALMTQSTDYLAVSLGQRATISCKASQSVDYDGDSYMNWYQQKPGQPPK
LLIYAASNLESGIPTRFSGSGFGTDFTLNIHPVEEEDAATYYCQQSNEDP
WTFGGGPKLEIK.

As one example of a suitable anti-C1s antibody, in some cases, the anti-C1s antibody comprises the following VH CDRs:

1) CDR-H1:
(SEQ ID NO: 39)
GFNIKDDYIHWV;
2) CDR-H2:
(SEQ ID NO: 40)
IDPADDHTKY;
and
3)
(SEQ ID NO: 41)
AIYGSGWAWFPY.

As one example of a suitable anti-C1s antibody, in some cases, the anti-C1s antibody comprises the following VL CDRs:

1) CDR-L1:
(SEQ ID NO: 42)
QSVDYDGDSYMN;
2) CDR-L2:
(SEQ ID NO: 43)
AASNLESGIP;
and
3) CDR-L3:
(SEQ ID NO: 44)
QQSNEDPWT.

As one example of a suitable anti-C1s antibody, in some cases, the anti-C1s antibody comprises the following VH CDRs and VL CDRs:

1) CDR-H1:
(SEQ ID NO: 39)
GFNIKDDYIHWV;
2) CDR-H2:
(SEQ ID NO: 40)
IDPADDHTKY;
3)
(SEQ ID NO: 41)
AIYGSGWAWFPY;
4) CDR-L1:
(SEQ ID NO: 42)
QSVDYDGDSYMN;
5) CDR-L2:
(SEQ ID NO: 43)
AASNLESGIP;
and
6) CDR-L3:
(SEQ ID NO: 44)
QQSNEDPWT.

As one example of a suitable anti-C1s antibody, in some cases, the anti-C1s antibody comprises VH CDRs present in the following VH amino acid sequence:

(SEQ ID NO: 112)
EVQLQQSGPELVKPGASVKISCKASGYSFTGYYIHWVKQSPEKSLEWIGE
INPTTNDTTYNQKFKAKATLTVDKSSNTAYMQLKSLTSEDSAVYYCSRDI
SGPAWFAYWGQGTLVTVSA.

As one example of a suitable anti-C1s antibody, in some cases, the anti-C1s antibody comprises VL CDRs present in the following VL amino acid sequence:

(SEQ ID NO: 113)
DIVLTQTTAIMSASPGEKVTMTCSASSSISYMYWFQQKPGTSPKRWIYDT
SKLASGVPARFSGSGSGTSYSLTISTMEAEDAATYYCHQRSSDPTFGGGT
KLEINR.

As one example of a suitable anti-C19 antibody, in some cases, the anti-C1s antibody comprises the following VH CDRs:

1) CDR-H1:
(SEQ ID NO: 45)
GYSFTGYYIHWV;
2) CDR-H2:
(SEQ ID NO: 46)
INPTTNDTTY;
and
3) CDR-H3:
(SEQ ID NO: 47)
SRDISGPAWFAY.

As one example of a suitable anti-C1s antibody, in some cases, the anti-C1s antibody comprises the following VL CDRs:

1) CDR-L1:
(SEQ ID NO: 48)
SSISYMYWFQQK;
2) CDR-L2:
(SEQ ID NO: 49)
DTSKLASGVP;
3) CDR-L3:
(SEQ ID NO: 50)
HQRSSDPT.

As one example of a suitable anti-C1s antibody, in some cases, the anti-C1s anti body comprises the following VH CDRs and VL CDRs:

1) CDR-H1:
(SEQ ID NO: 45)
GYSFTGYYIHWV;
2) CDR-H2:
(SEQ ID NO: 46)
INPTTNDTTY;
3) CDR-H3:
(SEQ ID NO: 47)
SRDISGPAWFAY;
4) CDR-L1:
(SEQ ID NO: 48)
SSISYMYWFQQK;
5) CDR-L2:
(SEQ ID NO: 49)
DTSKLASGVP;
and
6) CDR-L3:
(SEQ ID NO: 50)
HQRSSDPT.

As one example of a suitable anti-C1s antibody, in some cases, the anti-C1s antibody comprises VH CDRs present in the following VH amino acid sequence:

(SEQ ID NO: 114)
QVQLQQPGAELVRPGASVKLSCKVSGYTFTRYWMHWVKQRPGQGLEWIGE
INPSNSDTDYNEEFKSKATLTVDKSSSTAYMHLSSLTSEDSAVYYCTIDD
SVYGWFAYWGQGTLVTVSA.

As one example of a suitable anti-C1s antibody, in some cases, the anti-C1s antibody comprises VL CDRs present in the following VL amino acid sequence:

(SEQ ID NO: 115)
DIVMTQSPAIMFASPGERVTMTCSASSSISYMPWYPQKPGPSPKRWIYDT
SKLASGVPARFSGSGFGTFYSLTISSMEAEDAAPYYCHQRSSFPPFGAGT
KLELK.

As one example of a suitable anti-C1s antibody, in some cases, the anti-C1s antibody comprises the following VH CDRs:

(SEQ ID NO: 51)
1) CDR-H1: GYTFTRYWMHWV;
(SEQ ID NO: 52)
2) CDR-H2: INPSNSDTDY
and
(SEQ ID NO: 53)
3) CDR-H3: TIDDSVYGWFAY.

As one example of a suitable anti-C1s antibody, in some cases, the anti-C1s antibody comprises the following VL CDRs:

(SEQ ID NO: 54)
1) CDR-L1: SSISY;
(SEQ ID NO: 55)
2) CDR-L2: DTSKLASGVP;
and
(SEQ ID NO: 56)
3) CDR-L3: HQRSSFPP.

As one example of a suitable anti-C1s antibody, in some cases, the anti-C1s antibody comprises the following VH CDRs and VL CDRs:

(SEQ ID NO: 51)
1) CDR-H1: GYTFTRYWMHWV;
(SEQ ID NO: 52)
2) CDR-H2: INPSNSDTDY;
(SEQ ID NO: 53)
3) CDR-H3: TIDDSVYGWFAY;
(SEQ ID NO: 54)
4) CDR-L1: SSISY;
(SEQ ID NO: 55)
5) CDR-L2: DTSKLASGVP;
and
(SEQ ID NO: 56)
6) CDR-L3: HQRSSFPP.

As one example of a suitable anti-C1s antibody, in some cases, the anti-C1s antibody comprises VH CDRs present in the following VH amino acid sequence:

(SEQ ID NO: 116)
EVKLQQSGAELVRPGVSVKISCKVSGYTFTDYAMHCVKQSHAKSLEWIGV
ISIYNGDASYNQKFKDKATMTVDKSSSTSYMDLARLTSEESAVYNCVREA
PYLITTVFYAMDYWGQGTSVTVSS.

As one example of a suitable anti-C1s antibody, in some cases, the anti-C1s antibody comprises VL CDRs present in the following VL amino acid sequence:

(SEQ ID NO: 117)
DIVMTQSPAIMSASPGEKVTMTCSANSSISYMHWYQQKPGTSPKRWIYDT
SKLASGVPTRFSGSGSGTSYSLTISSMEAEDAATYYCHQRSFYLTFGSGT
KLEIK.

As one example of a suitable anti-C1s antibody, in some cases, the anti-C1s antibody comprises the following VH CDRs:

(SEQ ID NO: 57)
1) CDR-H1: GYTFTDYAMHCV;
(SEQ ID NO: 58)
2) CDR-H2: ISIYNGDASY;
and
(SEQ ID NO: 59)
3) CDR-H3: VREAPYLITTVFYAMDY.

As one example of a suitable anti-C1s antibody, in some cases, the anti-C1s antibody comprises the following VL CDRs:

(SEQ ID NO: 60)
1) CDR-L1: SSISYMHWYQQK;
(SEQ ID NO: 61)
2) CDR-L2: DTSKLASGVP;
and
(SEQ ID NO: 62)
3) CDR-L3: HQRSFYLT.

As one example of a suitable anti-C1s antibody, in some cases, the anti-C1s antibody comprises the following VH CDRs and V L CDRs:

(SEQ ID NO: 57)
1) CDR-H1: GYTFTDYAMHCV;
(SEQ ID NO: 58)
2) CDR-H2: ISIYNGDASY;
(SEQ ID NO: 59)
3) CDR-H3: VREAPYLITTVFYAMDY;
(SEQ ID NO: 60)
4) CDR-L1: SSISYMHWYQQK;
(SEQ ID NO: 61)
5) CDR-L2: DTSKLASGVP;
and
(SEQ ID NO: 62)
6) CDR-L3: HQRSFYLT.

As one example; of a suitable anti-C1s antibody, in some cases, the anti-C1s antibody comprises VH CDRs present in the following VH amino acid sequence:

(SEQ ID NO: 118)
QVQLQQSGAELVRPGASVKLSCKVSGYTFTRYWMHWVKQRPGQGLEWIGE
INPSNSDTDYNEEFKSKATLTVDKSSSTAYMHLSNLTSEDSAVYYCTIDD
SAYGWFAYWGQGTLVTVSA.

As one example of a suitable anti-C1s antibody, in some cases, the anti-C1s antibody comprises VL CDRs present in the following VL, amino acid sequence:

(SEQ ID NO: 119)
DIVLTQSTAIMSASPGERVTMTCSASSSISYMHWYHQKPGTSPKRWIYDT
SKLASGVPARFSGSGSGTSYSLAISSMEAEDAATYYCHQRSSFPTFGAGT
KLELK.

As one example of a suitable anti-C1s antibody, in some cases, the anti-C1s antibody comprises the following VH CDRs:

(SEQ ID NO: 63)
1) CDR-H1: GYTFTRYWMHWV;
(SEQ ID NO: 64)
2) CDR-H2: INPSNSDTDY;
and
(SEQ ID NO: 65)
3) CDR-H3: TIDDSAYGWFAY.

As one example of a suitable anti-C1s antibody, in some cases, the anti-C1s antibody comprises the following VL CDRs:

(SEQ ID NO: 66)
1) CDR-L1: SSISYMHWYHQK;
(SEQ ID NO: 67)
2) CDR-L2: DTSKLASGVP;
and
(SEQ ID NO: 68)
3) CDR-L3: HQRSSFPT.

As one example of a suitable anti-C1s antibody, in some cases, the anti-C1s antibody comprises the following VH CDRs and VL CDRs:

(SEQ ID NO: 63)
1) CDR-H1: GYTFTRYWMHWV;
(SEQ ID NO: 64)
2) CDR-H2: INPSNSDTDY;
(SEQ ID NO: 65)
3) CDR-H3: TIDDSAYGWFAY;
(SEQ ID NO: 66)
4) CDR-L1: SSISYMHWYHQK;
(SEQ ID NO: 67)
5) CDR-L2: DTSKLASGVP;
and
(SEQ ID NO: 68)
6) CDR-L3: HQRSSFPT.

As one example of u suitable anti-C1s antibody, in some cases, the anti-C1a antibody comprises VH CDRs present in the following VH amino acid sequence:

(SEQ ID NO: 120)
EVQLQQSGAELVRPGASVKLSCTASGFNIKDDYIHWVKQR
PEQGLEWIGRIDPADDHTKYAPKFQDKATMTADTSSNTAC
LQLNSLTSEDTAVYYCAIYGSGWAWFPYWGQGTLVSVSA.

As one example of a suitable anti-C1s antibody, in some cases, the anti-C1s antibody comprises VL CDRs present in the following V L amino acid sequence:

(SEQ ID NO: 121)
DIVLTQTPDYLAVSLGQRATISCKASQSVDYDGDSYMNWY
QQKPGQPPKLLIYAASNLESGIPARFSGSGSGTDFTLNIH
PVEEEDAATYYCQQSNEDPWTFGGGTKLEIK.

As one example of a suitable anti-C1s antibody, in some cases, the anti-C1s antibody comprises the following VH CDRs:

(SEQ ID NO: 69)
1) CDR-H1: GFNIKDDYIHWV;
(SEQ ID NO: 70)
2) CDR-H2: IDPADDHTKY;
and
(SEQ ID NO: 71)
3) CDR-H3: AIYGSGWAWFPY.

As one example of a suitable anti-C1s antibody, in some cases, the anti-C1s antibody comprises the following VL CDRs:

(SEQ. ID NO: 72)
1) CDR-L1: QSVDYDGDSYMN;
(SEQ ID NO: 73)
2) CDR-L2: AASNLESGIP;
and
(SEQ ID NO: 74)
3) CDR-L3: QQSNEDPWT.

As one example of a suitable anti-C1s antibody, in some cases, the anti-C1s antibody comprises the following VH CDRs and VL CDRs:

(SEQ ID NO: 69)
1) CDR-H1: GFNIKDDYIHWV;
(SEQ ID NO: 70)
2) CDR-H2: IDPADDHTKY;
(SEQ ID NO: 71)
3) CDR-H3: AIYGSGWAWFPY;
(SEQ ID NO: 72)
4) CDR-L1: QSVDYDGDSYMN;
(SEQ ID NO: 73)
5) CDR-L2: AASNLESGIP;
and
(SEQ ID NO: 74)
6) CDR-L3: QQSNEDPWT.

As one example of a suitable anti-C1s antibody, in some cases, the anti-C1s antibody comprises VH CDRs present in the following VH amino acid sequence:

(SEQ ID NO: 122)
EVQLQQSGPELVKPGASVKISCKASGYSFTGFYMQWVKQ
SPEKNLEWIGEINPTTGDETYNQKFQAKATLTVDKSSST
AYMQLKSLTSEDSAVYFCASDFYDGSFAWFEYWGKDYLT
VSA.

As one example of a suitable anti-C1s antibody, in some cases, the anti-C1s antibody comprises VL CDRs present in the following VL amino acid sequence:

(SEQ ID NO: 123)
DIVLTQSPVIMSASPGEKVTMTCSASSSISYIHWYQ
QKPGTSPKRWIYDTSKLASGVPARFSGSGSGTSYSLTI
SSMEAEDAATYYCHQRSSYLTFGSGTKLEIK.

As one example of a suitable anti-C1s antibody, in some cases, the anti-C1s antibody comprises the following VH CDRs:

(SEQ ID NO: 75)
1) CDR-H1: GYSFTGFYMQWV;
(SEQ ID NO: 76)
2) CDR-H2: INPTTGDETY;
and
(SEQ ID NO: 77)
3) CDR-H3: ASDFYDGSFAWFEY.

As one example of a suitable anti-C1s antibody, in some cases, the anti-C1s antibody comprises the following VL CDRs:

(SEQ ID NO: 78)
1) CDR-L1: SSISYIHWYQQK;
(SEQ ID NO: 79)
2) CDR-L2: DTSKLASGVP;
and
(SEQ ID NO: 80)
3) CDR-L3: HQRSSYLT.

As one example of a suitable anti-C1s antibody, in some cases, the anti-C1s antibody comprises the following VH CDRs and VL CDRs:

(SEQ ID NO: 75)
1) CDR-H1: GYSFTGFYMQWV;
(SEQ ID NO: 76)
2) CDR-H2: INPTTGDETY;
(SEQ ID NO: 77)
3) CDR-H3: ASDEYDGSFAWFEY;
(SEQ ID NO: 78)
4) CDR-L1: SSISYIHWYQQK;
(SEQ ID NO: 79)
5) CDR-L2: DTSKLASGVP;
and
(SEQ ID NO: 80)
6) CDR-L3: HQRSSYLT.

As one example of a suitable anti-C19 antibody, in some cases, the anti-C1s antibody comprise VH CDRs present in the following VH amino acid sequence:

(SEQ ID NO: 124)
QVKLQQSGPELVKPGTSVRISCKTSGYSFTGYYMH
WVKQSPEKSLEWIGEINPSIGDITYNQRFKAKATLT
VDKSSSTAYMQLKSLTSEDSAVYYCASDYYGGGFAWF
AYWGQGTLVTVSA.

As one example of a suitable anti-C1s antibody, in some cases, the anti-C1s antibody comprises VL CDRs present in the following VL amino acid sequence:

(SEQ ID NO: 125)
DIVMTQSPAIMSASSGEKVTMTCSASSSINYMHWYQQ
KPGTSPKRWIYDTSKLASGVPARFSGSGSGTSYSLTI
SSMEAEDTATYYCHQRSDSLTFGSGTKLEIK.

As one example of a suitable anti-C1s antibody, in some cases, the anti-C1s antibody comprises the following VH CDRs:

(SEQ ID NO: 81)
1) CDR-H1: GYSFTGYYMHWV;
(SEQ ID NO: 82)
2) CDR-H2: INPSIGDITY;
and
(SEQ ID NO: 83)
3) CDR-H3: ASDYYGGGFAWFAY.

As one example of a suitable anti-C1s antibody, in some cases, the anti-C1s antibody comprises the following VL CDRs:

(SEQ ID NO: 84)
1) CDR-L1: SSINYMHWYQQK;
(SEQ ID NO: 85)
2) CDR-L2: DTSKLASGVP;
and
(SEQ ID NO: 86)
3) CDR-L3: HQRSDSLT.

As one example of a suitable anti-C1s antibody, in some cases, the anti-C1s antibody comprises the following VH CDRs and VL CDRs:

(SEQ ID NO: 81)
1) CDR-H1: GYSFTGYYMHWV;
(SEQ ID NO: 82)
2) CDR-H2: INPSIGDITY;
(SEQ ID NO: 83)
3) CDR-H3: ASDYYGGGFAWFAY;
(SEQ ID NO: 84)
4) CDR-L1: SSINYMHWYQQK;
(SEQ ID NO: 85)
5) CDR-L2: DTSKLASGVP;
and
(SEQ ID NO: 86)
6) CDR-L3: HQRSDSLT.

As one example of a suitable anti-C1s antibody, in some cases, the anti-C1s antibody comprises the following VH CDRs:

(SEQ ID NO: 87)
1) CDR-H1: GFTFSNYAMSWV;
(SEQ ID NO: 88)
2) CDR-H2: ISSGGSHTYY;
and
(SEQ ID NO: 89)
3) CDR-H3: ARLFTGYAMDY.

As one example of a suitable anti-C1s is antibody, in some cases, the anti-C1s antibody comprises the following VL CDRs:

(SEQ ID NO: 90)
1) CDR-L1: SSVSSSYLHWYQ;
(SEQ ID NO: 91)
2) CDR-L2: STSNLASGVP;
and
(SEQ ID NO: 92)
3) CDR-L3: HQYYRLPPIT.

As one example of a suitable anti-C1s antibody, in some cases, the anti-C1s antibody comprises the following VH CDRs and VL CDRs:

(SEQ ID NO: 87)
1) CDR-H1: GFTFSNYAMSWV;
(SEQ ID NO: 88)
2) CDR-H2: ISSGGSHTYY;
(SEQ ID NO: 89)
3) CDR-H3: ARLFTGYAMDY;
(SEQ ID NO: 90)
4) CDR-L1: SSVSSSYLHWYQ;
(SEQ ID NO: 91)
5) CDR-L2: STSNLASGVP;
and
(SEQ ID NO: 92)
6) CDR-L3: HQYYRLPPIT.

As one example of a suitable anti-C1s antibody, in some cases, the anti-C1s antibody comprises VH CDRs present in the following VH amino acid sequence:

(SEQ ID NO: 93)
EVMLVESGGALVKPGGSLKLSCAASGFTFSNYAMSWVRQIPEKRLEWV
ATISSGGSHTYYLDSVKGRFTISRDNARDTLYLQMSSLRSEDTALYYC
ARLFTGYAMDYWGQGTSVTVSS

As one example of a suitable anti-C1s antibody, in some cases, the anti-C1s antibody comprises VL CDRs present in the following VL amino acid sequence:

(SEQ ID NO: 94)
QIVLTQSPAIMSASLGERVTMTCTASSSVSSSYLHWYQQKPGSSPKLW
IYSTSNLASGVPARFSGSGSGTFYSLTISSMEAEDDATYYCHQYYRLP
PITFGAGTKLELK.

As one example of a suitable anti-C1s antibody, in some cases, the anti-C1s antibody comprises the following VH CDRs:

(SEQ ID NO: 95)
1) CDR-H1: NYAMS;
(SEQ ID NO: 96)
2) CDR-H2: TISSGGSHTYYLDSVKG;
and
(SEQ ID NO: 97)
3) CDR-H3: LFTGYAMDY.

As one example of a suitable anti-C1s antibody, in some cases, the anti-C1s antibody comprises the following VL CDRs:

(SEQ ID NO: 98)
1) CDR-L1: TASSSVSSSYLH;
(SEQ ID NO: 99)
2) CDR-L2: STSNLAS;
and
(SEQ ID NO: 92)
3) CDR-L3: HQYYRLPPIT.

As one example of a suitable anti-C1s antibody, in some cases, the anti-C1s antibody comprises the following VH CDRs and VL CDRs:

(SEQ ID NO: 95)
1) CDR-H1: NYAMS;
(SEQ ID NO: 96)
2) CDR-H2: TISSGGSHTYYLDSVKG;
(SEQ ID NO: 97)
3) CDR-H3: LFTGYAMDY;
(SEQ ID NO: 98)
4) CDR-L1: TASSSVSSSYLH;
(SEQ ID NO: 99)
5) CDR-L2: STSNLAS;
and
(SEQ ID NO: 92)
6) CDR-L3: HQYYRLPPIT.

As noted above, in some cases, the anti-C1s antibody comprises a humanized V_{H} framework region. In some cases, the anti-C1s antibody comprises a humanized V_{L} framework region. In some cases, the anti-C1s antibody comprises a humanized V_{H} framework region and a humanized V_{L} framework region. Humanized V_{H} and V_{L} framework regions are known in the art, and can be readily generated by those skilled in the art. In some cases, a humanized V_{H} framework region is a consensus V_{H} framework region. In some cases, a humanized V_{L} framework region is a consensus V_{L} framework region.

Non-limiting examples of consensus human V_{H} framework regions suitable for use with V_{H} CDRs as described herein include (subgroup III consensus):

(SEQ ID NO: 126)
a) V_{H} FR1: EVQLVESGGGLVQPGGSLRLSCAAS;
(SEQ ID NO: 127)
b) V_{H} FR2: WVRQAPGKGLEWV;
(SEQ ID NO: 128)
c) V_{H} FR3: RFTISRDNSKNTLYLQMNSLRAEDTAVYYC;
and
(SEQ ID NO: 129)
d) V_{H} FR4: WGQGTLVTVSS.

In some cases, V_{H} FR3 comprises an amino acid substitution at position 71, 73, and/or 78; e.g., where the underlined and bolded R in RFTISRDNSKNTLYLQMNSLRAEDTAVYYC (SEQ ID NO:130) is amino acid 71 (Kabat numbering); the underlined and bolded N in RFTISRDNSKNTLYLQMNSLRAEDTAVYYC (SEQ ID NO:131) is amino acid 73 (Kabat numbering); and the underlined and bolded L in RFTISRDNSKNTLYLQMNSLRAEDTAVYYC (SEQ ID NO:132) is amino acid 78 (Kabat numbering). For example, in some cases, amino acid 71 is A; and/or amino acid 73 is T; and/or amino acid 78 is A. As an example, in some cases, a suitable consensus humanized V_{H} FR3 comprises the amino acid sequence: RFTISADTSKNTAYLQMNSLRAEDTAVYYC (SEQ ID NO: 133).

Non-limiting examples of consensus human V_{H} framework regions suitable for use with V_{H} CDRs as described herein include (subgroup I consensus):

(SEQ ID NO: 134)
a) V_{H} FR1: QVQLVQSGAEVKKPGASVKVSCKAS;
(SEQ ID NO: 135)
b) V_{H} FR2: WVRQAPGQGLEWM;
(SEQ ID NO: 136)
c) V_{H} FR3: RVTITADTSTSTAYMELSSLRSEDTAVYYC;
and
(SEQ ID NO: 137)
d) V_{H} FR4: WGQGTLVTVSS.

Non-limiting examples of consensus human V_{f}, framework regions suitable for use with V_{f} CDRs as described herein include (subgroup II consensus):

(SEQ ID NO: 138)
a) V_{H} FR1: QVQLQESGPGLVKPSQTLSLTCTVS;
(SEQ ID NO: 139)
b) V_{H} FR2: WIRQPPGKGLEWI;
(SEQ ID NO: 140)
c) V_{H} FR3: RVTISVDTSKNQFSLKLSSVTAADTAVYYC;
and
(SEQ ID NO: 141)
d) V_{H} FR4: WGQGTLVTVSS.

Non-limiting examples of consensus human Vₜ framework regions suitable for use with V_{L} CDRs as described herein include (subgroup I consensus):

(SEQ ID NO: 142)
a) V_{L} FR1: DIQMTQSPSSLSASVGDRVTITC;
(SEQ ID NO: 143)
b) V_{L} FR2: WYQQKPGKAPKLLIY;
(SEQ ID NO: 144)
c) V_{L} FR3: GVPSRFSGSGSGTDFTLTISSLQPEDFATYYC;
and
(SEQ ID NO: 145)
d) V_{L} FR4: FGQGTKVEIK.

Non-limiting examples of consensus human V_{L} framework regions suitable for use with V_{L} CDRs as described herein include (subgroup II consensus):

(SEQ ID NO: 146)
a) V_{L} FR1: DIVMTQSPLSLPVTPGEPASISC;
(SEQ ID NO: 147)
b) V_{L} FR2: WYLQKPGQSPQLLIY;
(SEQ ID NO: 148)
c) V_{L} FR3: GVPDRFSGSGSGTDFTLKISRVEAEDVGVYYC;
and
(SEQ ID NO: 149)
d) V_{L} FR4: FGQGTKVEIK.

Non-limiting examples of consensus human V_{L} framework regions suitable for use with V_{L} CDRs as described herein include (subgroup II consensus):

(SEQ ID NO: 150)
a) V_{L} FR1: DIVMTQSPDSLAVSLGERATINC;
(SEQ ID NO: 151)
b) V_{L} FR2: WYQQKPGQPPKLLIY;
(SEQ ID NO: 152)
c) V_{L} FR3: GVPDRFSGSGSGTDFTLTISSLQAEDFAVYYC;
and
(SEQ ID NO: 153)
d) V_{L} FR4: FGQGTKVEIK.

Non-limiting examples of consensus human Vₜ framework regions suitable for use with V_{L} CDRs as described herein include (subgroup IV consensus):

(SEQ ID NO: 154)
a) V_{L} FR1: DIVMTQSPDSLAVSLGERATINC;
(SEQ ID NO: 155)
b) V_{L} FR2: WYQQKPGQPPKLLIY;
(SEQ ID NO: 156)
c) V_{L} FR3: GVPDRFSGSGSGTDFTLTISSLQAEDFAVYYC;
and
(SEQ ID NO: 157)
d) V_{L} FR4: FGQGTKVEIK.

### Formulations

In carrying out a method of the present disclosure for treating an alloimmune or an autoimmune disorder, an anti-C1s antibody can be administered to an individual using any convenient means capable of resulting in the desired therapeutic effect. For example, an anti-C1s antibody can be formulated into pharmaceutical compositions by combination with appropriate, pharmaceutically acceptable carriers, pharmaceutically acceptable diluents, or other pharmaceutically acceptable excipients and can be formulated into preparations in solid, semi-solid, liquid or gaseous forms, such as tablets, capsules, powders, granules, ointments, solutions, suppositories, injections, inhalants and aerosols.

In pharmaceutical dosage forms, an anti-C1s antibody can be administered in the form of their pharmaceutically acceptable salts, or they can also be used alone or in appropriate association, as well as in combination, with other pharmaceutically active compounds. The following methods and excipients are merely exemplary and are in no way limiting.

For oral preparations, an anti-C1s antibody can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as tale or magnesium stearate; and if desired, with diluents, buffering agents, moistening agents, preservatives and flavoring agents.

An anti-C1s antibody can be formulated into preparations for injection by dissolving, suspending or emulsifying the antibody in an aqueous or nonaqueous solvent, such as vegetable or other similar oils, propylene glycol, synthetic aliphatic acid glycerides, injectable organic esters (e.g., ethyl oleate), esters of higher aliphatic acids or propylene glycol; and if desired, with conventional additives such as solubilizers, isotonic agents, suspending agents, emulsifying agents, stabilizers and preservatives. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Furthermore, the pharmaceutical composition of the present disclosure can comprise further agents such as dopamine or psychopharmacologic drugs, depending on the intended use of the pharmaceutical composition.

Pharmaceutical compositions comprising an anti-C1s antibody are prepared by mixing an anti-C1s antibody having the desired degree of purity with optional physiologically acceptable carriers, other excipients, stabilizers, surfactants, buffers and/or tonicity agents. Acceptable carriers, other excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine. N-methylglucosamine, galactosamine, and neuraminic acid; and/or non-ionic surfactants such as Tween. Brij Pluronics. Triton-X, or polyethylene glycol (PEG).

The pharmaceutical composition can be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents can be used for the production of pharmaceutical compositions for parenteral administration; see also Chen (1992) Drug Dev Ind Pharm 18, 1311-54.

Exemplary antibody concentrations in a pharmaceutical composition can range from about 1 mg/mL to about 200 mg/mL or from about 50 mg/mL to about 200 mg/mL, or from about 150 mg/mL to about 200 mg/mL.

An aqueous formulation of an anti-C1s antibody can be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent can be included in the antibody formulation to modulate the tonicity of the formulation. Exemplary tonicity agents include sodium chloride, potassium chloride, glycerin and any component from the group of amino acids, sugars as well as combinations thereof. In some embodiments, the aqueous formulation is isotonic, although hypertonic or hypotonic solutions can be suitable. The term “isotonic” denotes a solution having the same tonicity as some other solution with which it is compared, such as a physiological salt solution or serum. Tonicity agents can be used in an amount of about 5 mM to about 350 mM, e.g., in an amount of 100 mM to 350 mM.

A surfactant can also be added to the antibody formulation to reduce aggregation of the formulated antibody and/or minimize the formation of particulates in the formulation and/or reduce adsorption. Exemplary surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer. Pluronic), and % odium dodecyl sulfate (SDS). Examples of suitable polyoxyethylenesorbitan-fatty acid esters are polysorbate 20. (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Exemplary concentrations of surfactant can range from about 0.001% to about 1% w/v.

A lyoprotectant can also be added in order to protect the labile active ingredient (e.g. a protein) against destabilizing conditions during the lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, a formulation includes an anti-C1s antibody, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

For example, a formulation can be a liquid or lyophilized formulation suitable for parenteral administration, and can comprise: about 1 mg/mL to about 200 mg/mL of an anti-C1s antibody; about 0.001% to about 1% of at least one surfactant; about 1 mM to about 100 mM of a buffer; optionally about 10 mM to about 50) mM of a stabilizer; and about 5 mM to about 350 mM of a tonicity agent; and has a pH of about 4.0 to about 7.0.

### Methods of Monitoring Efficacy

The present disclosure provides a method of monitoring efficacy of a method of treating an alloimmune disorder or autoimmune disorder of the present disclosure. The method generally involves: a) detecting the level of autoantibody or alloantibody; and/or b) detecting the number of autoreactive or alloreactive B-cells; and/or c) detecting the level of a cytokine(s) produced by, or modulated by, a B-cell, in a biological sample obtained from an individual who has undergone treatment with a method of the present disclosure for treating an alloimmune disorder or autoimmune disorder. A change in (e.g., a decrease in) one or more of: a) the level of autoantibody or alloantibody; b) the number of autoreactive or alloreactive B-cells; and c) the level of a cytokine(s) produced by, or modulated by, a B-cell, compared to a pre-treatment level, or compared to a level or number in a sample taken at an earlier time point, indicates efficacy of the treatment. In some cases, the detecting is quantitative.

In some cases, a method of monitoring efficacy of a treatment method of the present disclosure comprises: a) detecting the level of autoantibody or alloantibody in a biological sample obtained at a first time point from an individual; and b) detecting the level of autoantibody or alloantibody in a biological sample obtained at a second time point from the individual. The second time point is later than the first time point. Where the level of autoantibody or alloantibody in the biological sample taken at the second time point is lower than the level of autoantibody or alloantibody in the biological sample taken at the first time point, efficacy of treatment is indicated. For alloantibodies (e.g. —anti-HLA antibodies), a switch in the isotype from a C1q-binding alloantibody to a non C1q-binding alloantibody would also demonstrate efficacy of treatment. Thus, in some cases, a method of monitoring efficacy of a treatment method of the present disclosure comprises: a) detecting the isotype of autoantibody or alloantibody in a biological sample obtained at a first time point from an individual; and b) detecting the isotype of autoantibody or alloantibody in a biological sample obtained at a second time point from the individual.

In some cases, a method of monitoring efficacy of a treatment method of the present disclosure comprises: a) detecting the level of (e.g., determining the number of) autoreactive B cells or alloreactive B cells in a biological sample obtained at a first time point from an individual; and b) detecting the level of (e.g., determining the number of) autoreactive B cells or alloreactive B cells in a biological sample obtained at a second time point from the individual. The second time point is later than the first time point. Where the level of autoreactive B cells or alloreactive B cells in the biological sample taken at the second time point is lower than the level of autoreactive B cells or alloreactive B cells in the biological sample taken at the first time point, efficacy of treatment is indicated.

in some cases, a method of monitoring efficacy of a treatment method of the present disclosure comprises: a) detecting the level of a cytokine(s) produced by, or modulated by, a B-cell, in a biological sample obtained at a first time point from an individual; and b) detecting the level of a cytokine(s) produced by, or modulated by, a B-cell, in a biological sample obtained at a second time point from the individual. The second time point is later than the first time point. Where the level of cytokine(s), produced by a B-cell, or modulated by a B-cell, in the biological sample taken at the second time point is altered compared to the level of cytokine(s), produced by a B-cell, or modulated by a B-cell, in the biological sample taken at the first time point, efficacy of treatment is indicated. For example, where the level of pro-inflammatory cytokine(s), produced by a B-cell, in the biological sample taken at the second time point is lower than the level of pro-inflammatory cytokine(s), produced by a B-cell, in the biological sample taken at the first time point, efficacy of treatment is indicated. Cytokines produced by B-cells include, e.g., pro-inflammatory cytokines such as IL-2, IL-4, IL-6, IL-12, IFN-γ, and TNF-α; and immunosuppressive cytokines such as IL-10 and TGF-β.

Suitable biological samples include, e.g., blood, serum, plasma, etc.

In some cases, the first time point is before the start of treatment with a method of the present disclosure for treating an alloimmune disorder or autoimmune disorder; and the second time point is after the start of treatment with a method of the present disclosure for treating an alloimmune disorder or autoimmune disorder. In some cases, the first time point is before the start of treatment with a method of the present disclosure for treating an alloimmune disorder or autoimmune disorder; and the second time point is from 2 days to 6 months (e.g., from 2 days to 7 days, from 1 week to 2 weeks, from 2 weeks to 4 weeks, from 1 month to 2 months, from 2 months to 3 months, from 3 months to 4 months, from 4 months to 5 months, or from 5 months to 6 months) after the start of treatment with a method of the present disclosure for treating an alloimmune disorder or autoimmune disorder.

In some cases, the first time point is after the start of treatment with a method of the present disclosure for treating an alloimmune disorder or autoimmune disorder. For example, in some cases, the first time point is from 2 days to 6 months (e.g., from 2 days to 7 days, from 1 week to 2 weeks, from 2 weeks to 4 weeks, from 1 month to 2 months, from 2 months to 3 months, from 3 months to 4 months, from 4 months to 5 months, or from 5 months to 6 months) after the start of treatment with a method of the present disclosure for treating an alloimmune disorder or autoimmune disorder; and the second time point is from 2 days to 6 months (e.g., from 2 days to 7 days, from 1 week to 2 weeks, from 2 weeks to 4 weeks, from 1 month to 2 months, from 2 months to 3 months, from 3 months to 4 months, from 4 months to 5 months, or from 5 months to 6 months) after the first time point.

Methods of determining the level of autoantibody or alloantibody are known in the art, and any known method can be used. Examples of suitable methods include immunological methods such as ELISA, LFIA. DIA, F-A. CLIA, CIA, MIA, RIA, and the like. For example, a detectably labeled autoantigen or alloantigen can be used in an assay to detect an autoantibody or alloantibody, respectively. Autoantibody present in a biological sample obtained from an individual being treated can be immobilized; and the detectably labeled autoantigen contacted with the immobilized autoantibody, forming a complex, where the presence or amount of detectable label indicates the presence or amount of autoantibody in the biological sample. Similarly, alloantibody present in a biological sample obtained from an individual being treated can be immobilized; and the detectably labeled alloantigen contacted with the immobilized alloantibody, forming a complex, where the presence or amount of detectable label indicates the presence or amount of alloantibody in the biological sample.

Methods of determining the level of (e.g., the number of) autoreactive B cells or alloreactive B cells are known in the art, and any known method can be used. Examples of suitable methods include flow cytometry, immunofluorescence, enzyme-linked immunospot (ELISPOT) assay, etc. The level of (e.g., the number of) autoreactive B cells or alloreactive B cells is determined in a sample obtained from an individual, where the sample can include, e.g., a tissue biopsy sample, blood, or bone marrow.

Methods of determining the level of a cytokine produced by, or modulated by, a B-cell are known in the art, and any known method can be used. Examples of suitable methods include, e.g., an ELISA assay.

### Subjects Suitable for Treatment

A variety of hosts (wherein the term “host” is used interchangeably herein with the terms “subject.” “individual,” and “patient”) are treatable according to the subject methods. Generally such hosts are “mammals” or “mammalian.” where these terms are used broadly to describe organisms which are within the class mammalia, including the orders carnivore (e.g., cats), herbivores (e.g., cattle, horses, and sheep), omnivores (e.g., dogs, goats, and pigs), rodentia (e.g., mice, guinea pigs, and rats), and primates (e.g., humans, chimpanzees, and monkeys). In some embodiments, the host is an individual that has a complement system, such as a mammal, fish, or invertebrate. In some embodiments, the host is a complement system-containing mammal, fish, or invertebrate companion animal, agricultural animal, work animal, zoo animal, or lab animal. In some embodiments, the host is human.

Individuals suitable for treatment using a method of the present disclosure for treating an autoimmune disorder include individuals having an autoimmune disorder mediated by autoantibodies. Individuals suitable for treatment using a method of the present disclosure for treating an autoimmune disorder include individuals having a disorder (e.g., diagnosed as having a disorder) such as Addison's disease, age-related macular degeneration, alopecia, autoimmune hepatitis (e.g., autoimmune hepatitis associated with hepatitis B virus infection; autoimmune hepatitis associated with hepatitis C virus infection), autoimmune hemolytic anemia, autoimmune skin diseases, autoimmune thyroid disease, bullous pemphigoid, celiac disease, cold agglutinin disease, dermatomyositis, type 1 diabetes mellitus. Grave's disease. Goodpasture's syndrome, Hashimoto's disease, hypoparathyroidism, hypopituitarism, hypothyroidism, idiopathic thrombocytopenic purpura, inflammatory bowel disease (e.g., Crohn's disease; ulcerative colitis), multiple sclerosis, myasthenia gravis, myocarditis, neuromyelitis optica, pemphigus vulgaris, pemphigus foliaceus, polymyositis, psoriasis, rheumatoid arthritis, sarcoidosis, scleroderma, Sjögren's syndrome, systemic lupus erythematosus, uveitis, and Wegener's granulomatosis and poly/dermatomyositis.

In some cases, the individual has been treated previously with a treatment regimen for an autoimmune disorder; and has failed to respond to the treatment. In some cases, the individual has been treated previously with a treatment regimen for an autoimmune disorder; and has relapsed, e.g., the autoimmune disorder has recurred.

In some cases, an individual that is suitable for treatment with a method of the present disclosure has a disease selected from age-related macular degeneration. Alzheimer's disease, amyotrophic lateral sclerosis, anaphylaxis, argyrophilic grain dementia, arthritis (e.g., rheumatoid arthritis), asthma, atherosclerosis, atypical hemolytic uremic syndrome, autoimmune diseases, autoimmune hemolytic anemia, Barraquer-Simons syndrome, Behçet's disease. British type amyloid angiopathy, bullous pemphigoid. Buerger's disease. C1q nephropathy, cancer, catastrophic antiphospholipid syndrome, cerebral amyloid angiopathy, cold agglutinin disease, corticobasal degeneration, Creutzfeldt-Jakob disease, Crohn's disease, cryoglobulinemic vasculitis, dementia pugilistica, dementia with Lewy Bodies (DLB), diffuse neurofibrillary tangles with calcification. Discoid lupus erythematosus. Down's syndrome, focal segmental glomerulosclerosis, formal thought disorder, frontotemporal dementia (FTD), frontotemporal dementia with parkinsonism linked to chromosome 17, frontotemporal lobar degeneration, Gerstmann-Straussler-Scheinker disease, Guillain-Barré syndrome. Hallervorden-Spatz disease, hemolytic-uremic syndrome, hereditary angioedema, hypophosphastasis, idiopathic pneumonia syndrome, immune complex diseases, inclusion body myositis, infectious disease (e.g., disease caused by bacterial (e.g., Neisseria meningitidis or Streptococcus) viral (e.g., human immunodeficiency virus (HIV)), or other infectious agents), inflammatory disease, ischemia/reperfusion injury, mild cognitive impairment, immunothrombocytopenic purpura (ITP), molybdenum cofactor deficiency (MoCD) type A, membranoproliferative glomerulonephritis (MPGN) I, membranoproliferative glomerulonephritis (MPGN) II (dense deposit disease), membranous nephritis, multi-infarct dementia, lupus (e.g., systemic lupus erythematosus (SLE)), glomerulonephritis, Kawasaki disease, multifocal motor neuropathy, multiple sclerosis, multiple system atrophy, myasthenia gravis, myocardial infarction, myotonic dystrophy, neuromyelitis optica, Niemann-Pick disease type C, non-Guamanian motor neuron disease with neurofibrillary tangles, Parkinson's disease. Parkinson's disease with dementia, paroxysmal nocturnal hemoglobinuria, Pemphigus vulgaris, Pick's disease, postencephalitic parkinsonism, polymyositis, prion protein cerebral amyloid angiopathy, progressive subcortical gliosis, progressive supranuclear palsy, psoriasis, sepsis. Shiga-toxin E coli (STEC)-HuS, spinal muscular atrophy, stroke, subacute sclerosing panencephalitis. Tangle only dementia, transplant rejection, vasculitis (e.g., ANCA associated vasculitis), Wegner's granulomatosis, sickle cell disease, cryoglobulinemia, mixed cryoglobulinemia, essential mixed cryoglobulinemia. Type II mixed cryoglobulinemia. Type II mixed cryoglobulinemia, nephritis, drug-induced thrombocytopenia, lupus nephritis, bullous pemphigoid, Epidermolysis bullosa acquisita, delayed hemolytic transfusion reaction, hypocomplementemic urticarial vasculitis syndrome, pseudophakic bullous keratopathy, and platelet refractoriness.

Individuals suitable for treatment using a method of the present disclosure for treating an alloimmune disorder include individuals who have received a donor organ or tissue, where such individuals are referred to as organ or tissue recipients. Individuals suitable for treatment using a method of the present disclosure for treating an alloimmune disorder include individuals who are to receive (e.g., who are scheduled to receive; who are on a wait list to receive; etc.) a donor organ or tissue, where such individuals are referred to as prospective organ or tissue recipients. Allograft organs and tissues include, but are not limited to, a kidney, a liver, a pancreas, a heart, a lung, skin, blood tissue (including whole blood; red blood cells; white blood cells; cord blood; and the like, where the blood tissue may comprise an isolated population of blood cells (buffy coat; red blood cells; platelets; lymphocytes; T cells; B cells; or some other population), or where the blood tissue comprises a mixed population of cells), small intestine, an endothelial tissue, a vascular tissue (e.g., a blood vessel), an eye, a stomach, a thymus, bone, bone marrow, cornea, a heart valve, an islet of Langerhans, or a tendon.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used. e.g., bp, base pair(s); kb, kilobase(s); pi, picoliter(s); s or sec, second(s); min. minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Materials and Methods

Preparation of ELISA plates. High-binding ELISA plates were coated overnight at +4° C. with 10 μg/mL of endotoxin-free plasma-derived whole human IgM (Hu IgM) or with mouse IgG raised against human IgM (MaH). The next day, plates were washed with phosphate-buffered saline (PBS) and blocked with 2% gelatin solution.

Deposition of complement. Normal human serum was diluted in GVB++ buffer to 2.5% for Hu IgM plates or to 5% for MaH plates and treated for 15-30 minutes at room temperature with: 1) 20-100 μg/mL of anti-C1s mouse monoclonal antibody TNT003, a humanized variant of TNT003, anti-C5 antibody, or matching isotype controls; 2) 350 μg/mL of anti-C1s mouse monoclonal antibody TNTO05, a humanized variant of TNT005, or matching isotype control; or 3) C3-immunodepleted human serum. Resulting serum solutions were incubated in corresponding plate for 90 minutes at 37° C. Complement deposition was stopped by 3 washes of room-temperature PBS.

Coating Human IgM plates with B-cell receptor (BCR) agonist. Hu IgM plates exposed to normal human scrum (NHS) were thoroughly washed and exposed to 15 μg/mL of goat anti-human IgM antibody F(ab′)2 fragment specific to Fc5μ for 30 minutes at room temperature. Plates were washed three times with PBS.

Activation of primary B cells. Normal primary human B-cells were pre-stained with Ca²⁺ flux reporter Fluo-4 according to a manufacturer's protocol. Stained cells were exposed to flu IgM or MaH-coated plates with deposited complement for 1 hour. Fluo-4 fluorescence was measured on Spectromax i3 instrument and Ca²⁺ flux values were determined as the area under the curve.

Proliferation of primary B cells. Carboxyfluorescein succinimidyl ester (CFSE)—pre-stained normal primary human B-cells were incubated in Mall-coated plates with deposited complement for 1 hour and then stimulated with TLR9 ligand CpG oligodeoxynucleotide (ODN) and kept in a CO₂ incubator. Eight days post stimulation, cells were fixed with 4% paraformaldehyde and stained with CD19-specific antibody conjugated with allophycocyanin (APC). Cells were analyzed by flow cytometry. The proliferating population of cells was identified as percent of CFSE^{low} cells in the intact single CD19 positive gate.

C3d, C5b ELISA. Hu IgM or Mall-coated plates with deposited complement were blocked in 1% casein and stained with either rabbit anti-human C3d or rabbit anti-human C5b primary antibody for 1 hour. Then, plates were thoroughly washed in PBS+TWEEN® 20 nonionic detergent (PBST) and stained with anti-rabbit antibody conjugated to horse radish peroxidase (HRP) for 1 hour. Plates were washed in PBST. The HRP signal was revealed using 3.3′,5,5′-tetramethylbenzidine (TMB) substrate. The reaction was stopped after 10 minutes with a low pH solution. C3d or C5b deposition was measured as optical density (OD) absorption at 405 nm on a plate reader and normalized to the levels of an appropriate isotype control.

### Results

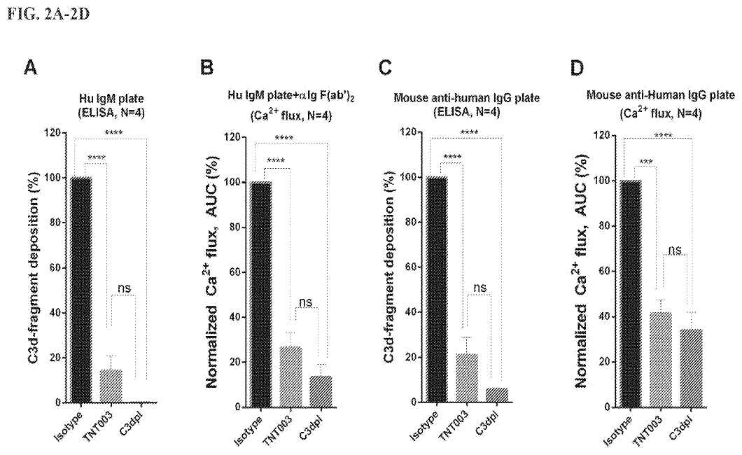

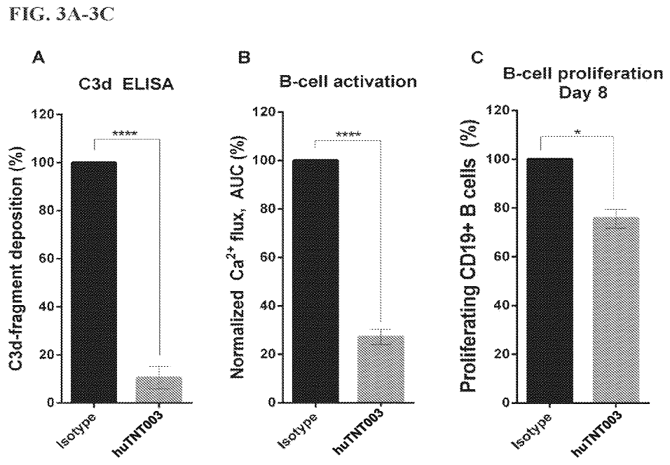

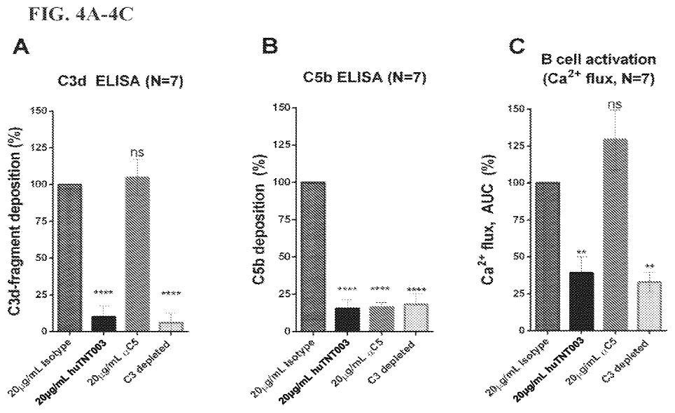

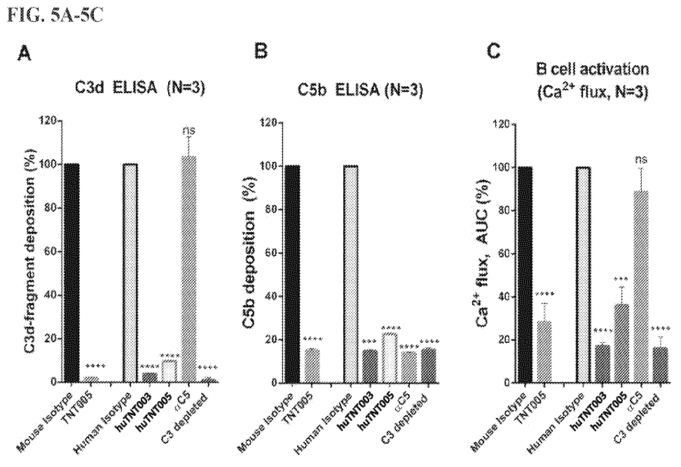

The results are depicted in FIG. 2A-2D. FIG. 3A-3C, FIG. 4A-4C, and FIG. 5A-SC.

As shown in FIG. 2A-2D. TNTO03, a mouse, monoclonal antibody that inhibits human C1s, prevents complement C3-mediated activation of normal primary human B-cells.

FIG. 2A-2D. (A). C3d ELISA. Deposition of complement C3d-fragment using normal human serum treated with an isotype control (mouse IgG2a). TNT003, or using C3-immunodepleted human serum (C3dpl) on human IgM-coated ELISA plates. (B). Activation of primary human B-cells. Calcium (Ca²⁺) flux in normal primary human B cells exposed to plates with deposited complement from (A) activated by the addition of B-cell receptor (BCR) agonist anti-Ig. (C). C3d ELISA. Deposition of complement C3d-fragment using normal human serum treated with an isotype control (mouse IgG2a). TNT003, or using C3-immunodepleted human serum on mouse IgG-coated ELISA plates. (D). Activation of primary human B-cells. Ca²⁺ flux in normal primary human B cells exposed to plates with deposited complement from (C). Mouse anti-human IgG is used as an immobilized BCR agonist. Values are normalized to matching isotype controls and are the average of four independent experiments on primary B cells derived from the blood of four separate human donors. Statistics were performed using one-way ANOVA (Tukey's multiple comparison test).

As shown in FIG. 3A-3C, a humanized variant of TNT003 (“huTNT003”), which is a humanized IgG4 monoclonal antibody that inhibits human C1s, prevents complement C3-mediated activation of normal primary human B cells.

FIG. 3A-3C. (A). C3d ELISA. Deposition of complement C3d-fragment using human serum treated with an isotype control (human IgG4) or a humanized variant of TNT003 on ELISA plates coated with B-cell receptor (BCR) agonist mouse IgG. (B). Activation of primary human B-cells exposed to deposited complement. Ca²⁺ flux in normal primary human B cells exposed to plates from (A). (C). Proliferation of primary human B-cells exposed to deposited complement. Normalized ratio of CFSE-low CD19+ B cells exposed to plates from (A) in the presence of Toll-like receptor 9 (TLR9) agonist CpG oligodeoxynucleotide (ODN) at day 8 post stimulation. Values are normalized to matching isotype controls and are the average of five independent experiments on primary B cells derived from the blood of five separate human donors. Statistics were performed using one-way ANOVA (Tukey's multiple comparison test).

As shown in FIG. 4A-4C, the C1s inhibitor (a humanized variant of TNT003), but not C5 inhibitor antibody, prevents complement C3-mediated activation of normal primary human B cells.

FIG. 4A-4C. (A). C3d ELISA. Deposition of complement C3d-fragment using human serum treated with an isotype control, a humanized variant of TNT003, C5 inhibitor antibody (αC5) or C3-depleted serum on ELISA plates coated with B-cell receptor (BCR) agonist mouse IgG. (B). C5b ELISA. Deposition of complement C5b using human serum treated with an isotype control, humanized variant of TNT003. C5 inhibitor antibody (αC5) or C3-depleted serum on ELISA plates coated with B-cell receptor (BCR) agonist mouse IgG. (C). Activation of primary human B-cells exposed to deposited complement. Ca²⁺ flux in normal primary human B cells exposed to plates from (A. B). Values are normalized to matching isotype controls and are the average of seven independent experiments on primary B cells derived from the blood of seven separate human donors. Statistics were performed using one-way ANOVA (Tukey's multiple comparison test). Shown statistics are the comparison with an appropriate isotype control. The difference in B cell response between the humanized variant of TNT003 and C3 depleted points is statistically non-significant (ns).

As shown in FIG. 5A-5C. C1s inhibitor antibodies with distinct modes of action, but not C5 inhibitor antibody, prevent complement C3-mediated activation of normal primary human B cells.

FIG. 5A-5C. (A). C3d ELISA. Deposition of complement C3d-fragment using human serum treated with mouse isotype controls. TNT005 (a mouse IgG2a monoclonal C1s inhibitor antibody with distinct mode of action), human isotype controls, humanized variant of TNT003, a humanized IgG4 version of TNT005. C5 inhibitor antibody (αC5) or C3-depleted scrum on ELISA plates coated with B-cell receptor (BCR) agonist mouse IgG. (B). C5b ELISA. Deposition of complement. C5b using human scrum treated as in (A). (C). Activation of primary human B-cells exposed to deposited complement. Ca²⁺ flux in normal primary human B cells exposed to plates from (A. B). Values are normalized to matching isotype controls and are the average of three independent experiments on primary B cells derived from the blood of three separate human donors. Statistics were performed using one-way ANOVA (Tukey's multiple comparison test). Shown statistics are the comparison with an appropriate isotype control. The difference in B cell response between the humanized variant of TNT003 and C3-depleted, between the humanized variant of TNT005 (“huTNT005”) and C3 depleted, and between the humanized variant of TNT005 and C3-depleted points is statistically non-significant (ns).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of reducing the level of autoantibody or alloantibody titers in an individual afflicted with an autoimmune or alloimmune disorder, the method comprising
administering to the individual an antibody that specifically binds complement component 1s (C1s) in an amount and for a period effective to reduce the level of autoantibody or alloantibody titers.

2-9. (canceled)

10. A method of reducing B-cell proliferation and/or B-cell activation in an individual afflicted with an autoimmune or alloimmune disorder, the method comprising
administering to the individual an antibody that specifically binds complement component 1s (C1s) in an amount and for a period effective to reduce B-cell proliferation.

11-35. (canceled)

36. A method of monitoring efficacy of treatment with an antibody that specifically binds complement component 1s (C1s) in an individual afflicted with an autoimmune or alloimmune disorder, the method comprising:
detecting a first level of at least one marker of B-cell activation in a first sample obtained from the individual at a first time point; and
detecting a second level of the at least one marker of B-cell activation in a second sample obtained from the individual at a second time point that is later than the first time point,
wherein a decrease in the second level compared to the first level of the at least one marker of B-cell activation indicates that treatment of the individual with the antibody is efficacious for treating the autoimmune or alloimmune disorder.

37. The method of claim 36, wherein the first time point is prior to treatment of the individual with the antibody, and the second time point is after treatment of the individual with the antibody.

38. The method of claim 36, wherein the first time point is after treatment of the individual with the antibody, and the second time point is from 2 days to 6 months after the first time point.

39. The method of claim 36, wherein the at least one marker of B-cell activation comprises at least one cytokine produced or modulated by a B-cell.

40. The method of claim 39, wherein the at least one cytokine comprises at least one pro-inflammatory cytokine and/or at least one immunosuppressive cytokine.

41. The method of claim 39, wherein the at least one cytokine is selected from the group consisting of IL-2, IL-4, IL-6, IL-12, IFN-7, TNF-α, IL-10, and TGF-β.

42. The method of claim 36, wherein the at least one marker of B-cell activation comprises at least one cell surface marker of a B-cell.

43. The method of claim 42, wherein the at least one cell surface marker is selected from the group consisting of CD23, CD25, CD27, CD30, CD38, CD69, CD80, CD86, CD135, IgM, and CD10.

44. The method of claim 36, wherein the first and second samples are biological samples selected from the group consisting of blood samples, serum samples, plasma samples, bone marrow samples, and tissue biopsy samples.

45. The method of claim 36, wherein the antibody is humanized.

46. The method of claim 45, wherein the antibody comprises a humanized VL framework region.

47. The method of claim 45, wherein the antibody comprises a humanized VH framework region.

48. The method of claim 45, wherein the antibody comprises a humanized VL framework region and a humanized VH framework region.

49. The method of claim 36, wherein the antibody comprises:
a heavy chain variable region comprising: a complementarity determining region (CDR)-H1 comprising amino acid sequence GFNIKDDYIHWV (SEQ ID NO: 9), a CDR-H2 comprising amino acid sequence IDPADGHTKY (SEQ ID NO: 10), and a CDR-H3 comprising amino acid sequence ARYGYGREVFDY (SEQ ID NO: 11); and
a light chain variable region comprising: a CDR-L1 comprising amino acid sequence QSVDYDGDSYMN (SEQ ID NO: 12), a CDR-L2 comprising amino acid sequence DASNLESGIP (SEQ ID NO: 13), and a CDR-L3 comprising amino acid sequence QQSNEDPWT (SEQ ID NO: 14).

50. The method of claim 36, wherein the antibody comprises:
a heavy chain variable region comprising: a CDR-H1 comprising amino acid sequence NYAMS (SEQ ID NO: 95), a CDR-H2 comprising amino acid sequence TISSGGSHTYYLDSVKG (SEQ ID NO: 96), and a CDR-H3 comprising amino acid sequence LFTGYAMDY (SEQ ID NO: 97); and
a light chain variable region comprising: a CDR-L1 comprising amino acid sequence TASSSVSSSYLH (SEQ ID NO: 98), a CDR-L2 comprising amino acid sequence STSNLAS (SEQ ID NO: 99), and a CDR-L3 comprising amino acid sequence HQYYRLPPIT (SEQ ID NO: 92).


SEQUENCE LISTING (158 sequences)

SEQ ID NO: 1 - protein, 12 aa, synthetic construct
  REGION 1..12: note = Synthetic Amino Acid Sequence
GFNIKDDYIHWV

SEQ ID NO: 2 - protein, 10 aa, synthetic construct
  REGION 1..10: note = Synthetic Amino Acid Sequence
IDPADDHTKY

SEQ ID NO: 3 - protein, 12 aa, synthetic construct
  REGION 1..12: note = Synthetic Amino Acid Sequence
AIYGSGWAWFPY

SEQ ID NO: 4 - protein, 12 aa, synthetic construct
  REGION 1..12: note = Synthetic Amino Acid Sequence
QSVDYDGDSYMN

SEQ ID NO: 5 - protein, 10 aa, synthetic construct
  REGION 1..10: note = Synthetic Amino Acid Sequence
AASNLESGIP

SEQ ID NO: 6 - protein, 9 aa, synthetic construct
  REGION 1..9: note = Synthetic Amino Acid Sequence
QQSNEDPWT

SEQ ID NO: 7 - protein, 119 aa, synthetic construct
  REGION 1..119: note = Synthetic Amino Acid Sequence
EVKLQQSGAELVRPGASVKLSCTASGFNIKDDYIHWVKQRPEQGLEWIGRIDPADGHTKYAPKFQVKATITADTSSNTAYLQLSSLTSEDTAVYYCARYGYGREVFDYWGQGTTLTVSS

SEQ ID NO: 8 - protein, 111 aa, synthetic construct
  REGION 1..111: note = Synthetic Amino Acid Sequence
DIVLTQSTDYLAVSLGQRATISCKASQSVDYDGDSYMNWYQQKPGQPPKLLIYAASNLESGIPARFSGSGSGTDFTLNIHPVEEEDAATYYCQQSNEDPWTFGGGTKLEIK

SEQ ID NO: 9 - protein, 12 aa, synthetic construct
  REGION 1..12: note = Synthetic Amino Acid Sequence
GFNIKDDYIHWV

SEQ ID NO: 10 - protein, 10 aa, synthetic construct
  REGION 1..10: note = Synthetic Amino Acid Sequence
IDPADGHTKY

SEQ ID NO: 11 - protein, 12 aa, synthetic construct
  REGION 1..12: note = Synthetic Amino Acid Sequence
ARYGYGREVFDY

SEQ ID NO: 12 - protein, 12 aa, synthetic construct
  REGION 1..12: note = Synthetic Amino Acid Sequence
QSVDYDGDSYMN

SEQ ID NO: 13 - protein, 10 aa, synthetic construct
  REGION 1..10: note = Synthetic Amino Acid Sequence
DASNLESGIP

SEQ ID NO: 14 - protein, 9 aa, synthetic construct
  REGION 1..9: note = Synthetic Amino Acid Sequence
QQSNEDPWT

SEQ ID NO: 15 - protein, 12 aa, synthetic construct
  REGION 1..12: note = Synthetic Amino Acid Sequence
GYTFTRYWMHWV

SEQ ID NO: 16 - protein, 10 aa, synthetic construct
  REGION 1..10: note = Synthetic Amino Acid Sequence
INPSNSDTDY

SEQ ID NO: 17 - protein, 12 aa, synthetic construct
  REGION 1..12: note = Synthetic Amino Acid Sequence
TIDDSAYGWFAY

SEQ ID NO: 18 - protein, 12 aa, synthetic construct
  REGION 1..12: note = Synthetic Amino Acid Sequence
SSISYMHWYHQK

SEQ ID NO: 19 - protein, 10 aa, synthetic construct
  REGION 1..10: note = Synthetic Amino Acid Sequence
DTSKLASGVP

SEQ ID NO: 20 - protein, 8 aa, synthetic construct
  REGION 1..8: note = Synthetic Amino Acid Sequence
HQRSSFPT

SEQ ID NO: 21 - protein, 12 aa, synthetic construct
  REGION 1..12: note = Synthetic Amino Acid Sequence
GYTFTRYWMHWV

SEQ ID NO: 22 - protein, 10 aa, synthetic construct
  REGION 1..10: note = Synthetic Amino Acid Sequence
INPSNSDTDY

SEQ ID NO: 23 - protein, 12 aa, synthetic construct
  REGION 1..12: note = Synthetic Amino Acid Sequence
TIDDSVYGWFAY

SEQ ID NO: 24 - protein, 12 aa, synthetic construct
  REGION 1..12: note = Synthetic Amino Acid Sequence
SSISYMHWYHQK

SEQ ID NO: 25 - protein, 10 aa, synthetic construct
  REGION 1..10: note = Synthetic Amino Acid Sequence
DTSKLASGVP

SEQ ID NO: 26 - protein, 8 aa, synthetic construct
  REGION 1..8: note = Synthetic Amino Acid Sequence
HQRSSFPT

SEQ ID NO: 27 - protein, 12 aa, synthetic construct
  REGION 1..12: note = Synthetic Amino Acid Sequence
GFNIKDDYIHWV

SEQ ID NO: 28 - protein, 10 aa, synthetic construct
  REGION 1..10: note = Synthetic Amino Acid Sequence
IDPADDHTKY

SEQ ID NO: 29 - protein, 12 aa, synthetic construct
  REGION 1..12: note = Synthetic Amino Acid Sequence
AIYGSGWAWFPY

SEQ ID NO: 30 - protein, 12 aa, synthetic construct
  REGION 1..12: note = Synthetic Amino Acid Sequence
QSVDYDGDSYMN

SEQ ID NO: 31 - protein, 10 aa, synthetic construct
  REGION 1..10: note = Synthetic Amino Acid Sequence
AASNLEFGIP

SEQ ID NO: 32 - protein, 9 aa, synthetic construct
  REGION 1..9: note = Synthetic Amino Acid Sequence
QQSNEDPWT

SEQ ID NO: 33 - protein, 12 aa, synthetic construct
  REGION 1..12: note = Synthetic Amino Acid Sequence
GFNIKDDYIHWV

SEQ ID NO: 34 - protein, 10 aa, synthetic construct
  REGION 1..10: note = Synthetic Amino Acid Sequence
IDPADGHTKY

SEQ ID NO: 35 - protein, 12 aa, synthetic construct
  REGION 1..12: note = Synthetic Amino Acid Sequence
ARYGYGREVFDY

SEQ ID NO: 36 - protein, 12 aa, synthetic construct
  REGION 1..12: note = Synthetic Amino Acid Sequence
QSVDYDGDSYMN

SEQ ID NO: 37 - protein, 10 aa, synthetic construct
  REGION 1..10: note = Synthetic Amino Acid Sequence
DASNLEFGIP

SEQ ID NO: 38 - protein, 9 aa, synthetic construct
  REGION 1..9: note = Synthetic Amino Acid Sequence
QQSNEDPWT

SEQ ID NO: 39 - protein, 12 aa, synthetic construct
  REGION 1..12: note = Synthetic Amino Acid Sequence
GFNIKDDYIHWV

SEQ ID NO: 40 - protein, 10 aa, synthetic construct
  REGION 1..10: note = Synthetic Amino Acid Sequence
IDPADDHTKY

SEQ ID NO: 41 - protein, 12 aa, synthetic construct
  REGION 1..12: note = Synthetic Amino Acid Sequence
AIYGSGWAWFPY

SEQ ID NO: 42 - protein, 12 aa, synthetic construct
  REGION 1..12: note = Synthetic Amino Acid Sequence
QSVDYDGDSYMN

SEQ ID NO: 43 - protein, 10 aa, synthetic construct
  REGION 1..10: note = Synthetic Amino Acid Sequence
AASNLESGIP

SEQ ID NO: 44 - protein, 9 aa, synthetic construct
  REGION 1..9: note = Synthetic Amino Acid Sequence
QQSNEDPWT

SEQ ID NO: 45 - protein, 12 aa, synthetic construct
  REGION 1..12: note = Synthetic Amino Acid Sequence
GYSFTGYYIHWV

SEQ ID NO: 46 - protein, 10 aa, synthetic construct
  REGION 1..10: note = Synthetic Amino Acid Sequence
INPTTNDTTY

SEQ ID NO: 47 - protein, 12 aa, synthetic construct
  REGION 1..12: note = Synthetic Amino Acid Sequence
SRDISGPAWFAY

SEQ ID NO: 48 - protein, 12 aa, synthetic construct
  REGION 1..12: note = Synthetic Amino Acid Sequence
SSISYMYWFQQK

SEQ ID NO: 49 - protein, 10 aa, synthetic construct
  REGION 1..10: note = Synthetic Amino Acid Sequence
DTSKLASGVP

SEQ ID NO: 50 - protein, 8 aa, synthetic construct
  REGION 1..8: note = Synthetic Amino Acid Sequence
HQRSSDPT

SEQ ID NO: 51 - protein, 12 aa, synthetic construct
  REGION 1..12: note = Synthetic Amino Acid Sequence
GYTFTRYWMHWV

SEQ ID NO: 52 - protein, 10 aa, synthetic construct
  REGION 1..10: note = Synthetic Amino Acid Sequence
INPSNSDTDY

SEQ ID NO: 53 - protein, 12 aa, synthetic construct
  REGION 1..12: note = Synthetic Amino Acid Sequence
TIDDSVYGWFAY

SEQ ID NO: 54 - protein, 5 aa, synthetic construct
  REGION 1..5: note = Synthetic Amino Acid Sequence
SSISY

SEQ ID NO: 55 - protein, 10 aa, synthetic construct
  REGION 1..10: note = Synthetic Amino Acid Sequence
DTSKLASGVP

SEQ ID NO: 56 - protein, 8 aa, synthetic construct
  REGION 1..8: note = Synthetic Amino Acid Sequence
HQRSSFPP

SEQ ID NO: 57 - protein, 12 aa, synthetic construct
  REGION 1..12: note = Synthetic Amino Acid Sequence
GYTFTDYAMHCV

SEQ ID NO: 58 - protein, 10 aa, synthetic construct
  REGION 1..10: note = Synthetic Amino Acid Sequence
ISIYNGDASY

SEQ ID NO: 59 - protein, 17 aa, synthetic construct
  REGION 1..17: note = Synthetic Amino Acid Sequence
VREAPYLITTVFYAMDY

SEQ ID NO: 60 - protein, 12 aa, synthetic construct
  REGION 1..12: note = Synthetic Amino Acid Sequence
SSISYMHWYQQK

SEQ ID NO: 61 - protein, 10 aa, synthetic construct
  REGION 1..10: note = Synthetic Amino Acid Sequence
DTSKLASGVP

SEQ ID NO: 62 - protein, 8 aa, synthetic construct
  REGION 1..8: note = Synthetic Amino Acid Sequence
HQRSFYLT

SEQ ID NO: 63 - protein, 12 aa, synthetic construct
  REGION 1..12: note = Synthetic Amino Acid Sequence
GYTFTRYWMHWV

SEQ ID NO: 64 - protein, 10 aa, synthetic construct
  REGION 1..10: note = Synthetic Amino Acid Sequence
INPSNSDTDY

SEQ ID NO: 65 - protein, 12 aa, synthetic construct
  REGION 1..12: note = Synthetic Amino Acid Sequence
TIDDSAYGWFAY

SEQ ID NO: 66 - protein, 12 aa, synthetic construct
  REGION 1..12: note = Synthetic Amino Acid Sequence
SSISYMHWYHQK

SEQ ID NO: 67 - protein, 10 aa, synthetic construct
  REGION 1..10: note = Synthetic Amino Acid Sequence
DTSKLASGVP

SEQ ID NO: 68 - protein, 8 aa, synthetic construct
  REGION 1..8: note = Synthetic Amino Acid Sequence
HQRSSFPT

SEQ ID NO: 69 - protein, 12 aa, synthetic construct
  REGION 1..12: note = Synthetic Amino Acid Sequence
GFNIKDDYIHWV

SEQ ID NO: 70 - protein, 10 aa, synthetic construct
  REGION 1..10: note = Synthetic Amino Acid Sequence
IDPADDHTKY

SEQ ID NO: 71 - protein, 12 aa, synthetic construct
  REGION 1..12: note = Synthetic Amino Acid Sequence
AIYGSGWAWFPY

SEQ ID NO: 72 - protein, 12 aa, synthetic construct
  REGION 1..12: note = Synthetic Amino Acid Sequence
QSVDYDGDSYMN

SEQ ID NO: 73 - protein, 10 aa, synthetic construct
  REGION 1..10: note = Synthetic Amino Acid Sequence
AASNLESGIP

SEQ ID NO: 74 - protein, 9 aa, synthetic construct
  REGION 1..9: note = Synthetic Amino Acid Sequence
QQSNEDPWT

SEQ ID NO: 75 - protein, 12 aa, synthetic construct
  REGION 1..12: note = Synthetic Amino Acid Sequence
GYSFTGFYMQWV

SEQ ID NO: 76 - protein, 10 aa, synthetic construct
  REGION 1..10: note = Synthetic Amino Acid Sequence
INPTTGDETY

SEQ ID NO: 77 - protein, 14 aa, synthetic construct
  REGION 1..14: note = Synthetic Amino Acid Sequence
ASDFYDGSFAWFEY

SEQ ID NO: 78 - protein, 12 aa, synthetic construct
  REGION 1..12: note = Synthetic Amino Acid Sequence
SSISYIHWYQQK

SEQ ID NO: 79 - protein, 10 aa, synthetic construct
  REGION 1..10: note = Synthetic Amino Acid Sequence
DTSKLASGVP

SEQ ID NO: 80 - protein, 8 aa, synthetic construct
  REGION 1..8: note = Synthetic Amino Acid Sequence
HQRSSYLT

SEQ ID NO: 81 - protein, 12 aa, synthetic construct
  REGION 1..12: note = Synthetic Amino Acid Sequence
GYSFTGYYMHWV

SEQ ID NO: 82 - protein, 10 aa, synthetic construct
  REGION 1..10: note = Synthetic Amino Acid Sequence
INPSIGDITY

SEQ ID NO: 83 - protein, 14 aa, synthetic construct
  REGION 1..14: note = Synthetic Amino Acid Sequence
ASDYYGGGFAWFAY

SEQ ID NO: 84 - protein, 12 aa, synthetic construct
  REGION 1..12: note = Synthetic Amino Acid Sequence
SSINYMHWYQQK

SEQ ID NO: 85 - protein, 10 aa, synthetic construct
  REGION 1..10: note = Synthetic Amino Acid Sequence
DTSKLASGVP

SEQ ID NO: 86 - protein, 8 aa, synthetic construct
  REGION 1..8: note = Synthetic Amino Acid Sequence
HQRSDSLT

SEQ ID NO: 87 - protein, 12 aa, synthetic construct
  REGION 1..12: note = Synthetic Amino Acid Sequence
GFTFSNYAMSWV

SEQ ID NO: 88 - protein, 10 aa, synthetic construct
  REGION 1..10: note = Synthetic Amino Acid Sequence
ISSGGSHTYY

SEQ ID NO: 89 - protein, 11 aa, synthetic construct
  REGION 1..11: note = Synthetic Amino Acid Sequence
ARLFTGYAMDY

SEQ ID NO: 90 - protein, 12 aa, synthetic construct
  REGION 1..12: note = Synthetic Amino Acid Sequence
SSVSSSYLHWYQ

SEQ ID NO: 91 - protein, 10 aa, synthetic construct
  REGION 1..10: note = Synthetic Amino Acid Sequence
STSNLASGVP

SEQ ID NO: 92 - protein, 10 aa, synthetic construct
  REGION 1..10: note = Synthetic Amino Acid Sequence
HQYYRLPPIT

SEQ ID NO: 93 - protein, 118 aa, synthetic construct
  REGION 1..118: note = Synthetic Amino Acid Sequence
EVMLVESGGALVKPGGSLKLSCAASGFTFSNYAMSWVRQIPEKRLEWVATISSGGSHTYYLDSVKGRFTISRDNARDTLYLQMSSLRSEDTALYYCARLFTGYAMDYWGQGTSVTVSS

SEQ ID NO: 94 - protein, 109 aa, synthetic construct
  REGION 1..109: note = Synthetic Amino Acid Sequence
QIVLTQSPAIMSASLGERVTMTCTASSSVSSSYLHWYQQKPGSSPKLWIYSTSNLASGVPARFSGSGSGTFYSLTISSMEAEDDATYYCHQYYRLPPITFGAGTKLELK

SEQ ID NO: 95 - protein, 5 aa, synthetic construct
  REGION 1..5: note = Synthetic Amino Acid Sequence
NYAMS

SEQ ID NO: 96 - protein, 17 aa, synthetic construct
  REGION 1..17: note = Synthetic Amino Acid Sequence
TISSGGSHTYYLDSVKG

SEQ ID NO: 97 - protein, 9 aa, synthetic construct
  REGION 1..9: note = Synthetic Amino Acid Sequence
LFTGYAMDY

SEQ ID NO: 98 - protein, 12 aa, synthetic construct
  REGION 1..12: note = Synthetic Amino Acid Sequence
TASSSVSSSYLH

SEQ ID NO: 99 - protein, 7 aa, synthetic construct
  REGION 1..7: note = Synthetic Amino Acid Sequence
STSNLAS

SEQ ID NO: 100 - protein, 119 aa, synthetic construct
  REGION 1..119: note = Synthetic Amino Acid Sequence
EVQLQQSGAELVRPGASVKLSCTASGFNIKDDYIHWVKQRPEQGLEWIGRIDPADDHTKYAPKFQDKATMTADTSSNTACLQLNSLTSEDTAVYYCAIYGSGWAWFPYWGQGTLVSVSA

SEQ ID NO: 101 - protein, 111 aa, synthetic construct; identical to SEQ ID NO: 8
  REGION 1..111: note = Synthetic Amino Acid Sequence

SEQ ID NO: 102 - protein, 119 aa, synthetic construct
  REGION 1..119: note = Synthetic Amino Acid Sequence
QVQLQQPGAELVRPGASVKLSCKVSGYTFTRYWMHWVKQRPGQGLEWIGEINPSNSDTDYNEEFKSKATLTVDKSSSTAYMHLSSLTSEDSAVYYCTIDDSAYGWFAYWGQGTLVTVSA

SEQ ID NO: 103 - protein, 105 aa, synthetic construct
  REGION 1..105: note = Synthetic Amino Acid Sequence
DIVMTQSPAIMSASPGERVTMTCSASSSISYMHWYHQKPGTSPKRWIYDTSKLASGVPARFSGSGSGTSYSLTISSMEAEDAATYYCHQRSSFPTFGAGTKLELK

SEQ ID NO: 104 - protein, 119 aa, synthetic construct
  REGION 1..119: note = Synthetic Amino Acid Sequence
QVQLQQPGAELVRPGASVKLSCKVSGYTFTRYWMHWVKQRPGQGLEWIGEINPSNSDTDYNEEFKSKATLTVDKSSSTAYMHLSSLTSEDSAVYYCTIDDSVYGWFAYWGQGTLVTVSA

SEQ ID NO: 105 - protein, 105 aa, synthetic construct
  REGION 1..105: note = Synthetic Amino Acid Sequence
DIVITQSPAIMSASPGERVTMTCSASSSISYMHWYHQKPGTSPKRWIYDTSKLASGVPARFSGSGSGTSYSLTISSMEAEDAATYYCHQRSSFPTFGAGTKLELK

SEQ ID NO: 106 - protein, 149 aa, synthetic construct
  REGION 1..149: note = Synthetic Amino Acid Sequence
QVQLQQSGAELVRPGASVKLSCTASGFNIKDDYIHWVKQRPEQGLEWIGRIDPADDHTKYAPKFQDKATMTADTSSNTACLQLNSLTSEDTAVYYCAIYGSGWAWFPYWGQGTLVSVSAAKTTAPSVYPLAPVCGDTTGSSVTLGCLVK

SEQ ID NO: 107 - protein, 111 aa, synthetic construct
  REGION 1..111: note = Synthetic Amino Acid Sequence
DIVMTQSPDYLAVSLGQRAPISCKASQSVDYDGDSYMNWYQQKPGQPPKLLIYAASNLEFGIPTRFSGSGFGTDFPLNIHPVEEEDAATYYCQQSNEDPWTFGGGPKLEIK

SEQ ID NO: 108 - protein, 119 aa, synthetic construct; identical to SEQ ID NO: 7
  REGION 1..119: note = Synthetic Amino Acid Sequence

SEQ ID NO: 109 - protein, 111 aa, synthetic construct
  REGION 1..111: note = Synthetic Amino Acid Sequence
DIVLTQFPTFLAVFLGQRAPISCKASQSVDYDGDSYMNWFQQKTGQPPKILIYDASNLEFGIPTRFSGSGFGTDFPLNIHPVEEEDAAIYFCQQSNEDPWTFGGGPKLEIK

SEQ ID NO: 110 - protein, 119 aa, synthetic construct
  REGION 1..119: note = Synthetic Amino Acid Sequence
EVKLEQSGAELVRPGASVKLSCTASGFNIKDDYIHWVKQRPEQGLEWIGRIDPADDHTKYAPKFQDKATMTADTSSNTACLQLNSLTSEDTAVYYCAIYGSGWAWFPYWGQGTLVSVSA

SEQ ID NO: 111 - protein, 112 aa, synthetic construct
  REGION 1..112: note = Synthetic Amino Acid Sequence
EFALMTQSTDYLAVSLGQRATISCKASQSVDYDGDSYMNWYQQKPGQPPKLLIYAASNLESGIPTRFSGSGFGTDFTLNIHPVEEEDAATYYCQQSNEDPWTFGGGPKLEIK

SEQ ID NO: 112 - protein, 119 aa, synthetic construct
  REGION 1..119: note = Synthetic Amino Acid Sequence
EVQLQQSGPELVKPGASVKISCKASGYSFTGYYIHWVKQSPEKSLEWIGEINPTTNDTTYNQKFKAKATLTVDKSSNTAYMQLKSLTSEDSAVYYCSRDISGPAWFAYWGQGTLVTVSA

SEQ ID NO: 113 - protein, 106 aa, synthetic construct
  REGION 1..106: note = Synthetic Amino Acid Sequence
DIVLTQTTAIMSASPGEKVTMTCSASSSISYMYWFQQKPGTSPKRWIYDTSKLASGVPARFSGSGSGTSYSLTISTMEAEDAATYYCHQRSSDPTFGGGTKLEINR

SEQ ID NO: 114 - protein, 119 aa, synthetic construct; identical to SEQ ID NO: 104
  REGION 1..119: note = Synthetic Amino Acid Sequence

SEQ ID NO: 115 - protein, 106 aa, synthetic construct
  REGION 1..106: note = Synthetic Amino Acid Sequence
VDIVMTQSPAIMFASPGERVTMTCSASSSISYMPWYPQKPGPSPKRWIYDTSKLASGVPARFSGSGFGTFYSLTISSMEAEDAAPYYCHQRSSFPPFGAGTKLELK

SEQ ID NO: 116 - protein, 124 aa, synthetic construct
  REGION 1..124: note = Synthetic Amino Acid Sequence
EVKLQQSGAELVRPGVSVKISCKVSGYTFTDYAMHCVKQSHAKSLEWIGVISIYNGDASYNQKFKDKATMTVDKSSSTSYMDLARLTSEESAVYNCVREAPYLITTVFYAMDYWGQGTSVTVSS

SEQ ID NO: 117 - protein, 105 aa, synthetic construct
  REGION 1..105: note = Synthetic Amino Acid Sequence
DIVMTQSPAIMSASPGEKVTMTCSANSSISYMHWYQQKPGTSPKRWIYDTSKLASGVPTRFSGSGSGTSYSLTISSMEAEDAATYYCHQRSFYLTFGSGTKLEIK

SEQ ID NO: 118 - protein, 119 aa, synthetic construct
  REGION 1..119: note = Synthetic Amino Acid Sequence
QVQLQQSGAELVRPGASVKLSCKVSGYTFTRYWMHWVKQRPGQGLEWIGEINPSNSDTDYNEEFKSKATLTVDKSSSTAYMHLSNLTSEDSAVYYCTIDDSAYGWFAYWGQGTLVTVSA

SEQ ID NO: 119 - protein, 105 aa, synthetic construct
  REGION 1..105: note = Synthetic Amino Acid Sequence
DIVLTQSTAIMSASPGERVTMTCSASSSISYMHWYHQKPGTSPKRWIYDTSKLASGVPARFSGSGSGTSYSLAISSMEAEDAATYYCHQRSSFPTFGAGTKLELK

SEQ ID NO: 120 - protein, 119 aa, synthetic construct; identical to SEQ ID NO: 100
  REGION 1..119: note = Synthetic Amino Acid Sequence

SEQ ID NO: 121 - protein, 111 aa, synthetic construct
  REGION 1..111: note = Synthetic Amino Acid Sequence
DIVLTQTPDYLAVSLGQRATISCKASQSVDYDGDSYMNWYQQKPGQPPKLLIYAASNLESGIPARFSGSGSGTDFTLNIHPVEEEDAATYYCQQSNEDPWTFGGGTKLEIK

SEQ ID NO: 122 - protein, 120 aa, synthetic construct
  REGION 1..120: note = Synthetic Amino Acid Sequence
EVQLQQSGPELVKPGASVKISCKASGYSFTGFYMQWVKQSPEKNLEWIGEINPTTGDETYNQKFQAKATLTVDKSSSTAYMQLKSLTSEDSAVYFCASDFYDGSFAWFEYWGKDYLTVSA

SEQ ID NO: 123 - protein, 105 aa, synthetic construct
  REGION 1..105: note = Synthetic Amino Acid Sequence
DIVLTQSPVIMSASPGEKVTMTCSASSSISYIHWYQQKPGTSPKRWIYDTSKLASGVPARFSGSGSGTSYSLTISSMEAEDAATYYCHQRSSYLTFGSGTKLEIK

SEQ ID NO: 124 - protein, 121 aa, synthetic construct
  REGION 1..121: note = Synthetic Amino Acid Sequence
QVKLQQSGPELVKPGTSVRISCKTSGYSFTGYYMHWVKQSPEKSLEWIGEINPSIGDITYNQRFKAKATLTVDKSSSTAYMQLKSLTSEDSAVYYCASDYYGGGFAWFAYWGQGTLVTVSA

SEQ ID NO: 125 - protein, 105 aa, synthetic construct
  REGION 1..105: note = Synthetic Amino Acid Sequence
DIVMTQSPAIMSASSGEKVTMTCSASSSINYMHWYQQKPGTSPKRWIYDTSKLASGVPARFSGSGSGTSYSLTISSMEAEDTATYYCHQRSDSLTFGSGTKLEIK

SEQ ID NO: 126 - protein, 25 aa, synthetic construct [sequence omitted]

SEQ ID NO: 127 - protein, 13 aa, synthetic construct
  REGION 1..13: note = Synthetic Amino Acid Sequence
WVRQAPGKGLEWV

SEQ ID NO: 128 - protein, 30 aa, synthetic construct
  REGION 1..30: note = Synthetic Amino Acid Sequence
RFTISRDNSKNTLYLQMNSLRAEDTAVYYC

SEQ ID NO: 129 - protein, 11 aa, synthetic construct
  REGION 1..11: note = Synthetic Amino Acid Sequence
WGQGTLVTVSS

SEQ ID NO: 130 - protein, 30 aa, synthetic construct; identical to SEQ ID NO: 128
  REGION 1..30: note = Synthetic Amino Acid Sequence

SEQ ID NO: 131 - protein, 30 aa, synthetic construct; identical to SEQ ID NO: 128
  REGION 1..30: note = Synthetic Amino Acid Sequence

SEQ ID NO: 132 - protein, 30 aa, synthetic construct; identical to SEQ ID NO: 128
  REGION 1..30: note = Synthetic Amino Acid Sequence

SEQ ID NO: 133 - protein, 30 aa, synthetic construct
  REGION 1..30: note = Synthetic Amino Acid Sequence
RFTISADTSKNTAYLQMNSLRAEDTAVYYC

SEQ ID NO: 134 - protein, 25 aa, synthetic construct [sequence omitted]

SEQ ID NO: 135 - protein, 13 aa, synthetic construct
  REGION 1..13: note = Synthetic Amino Acid Sequence
WVRQAPGQGLEWM

SEQ ID NO: 136 - protein, 30 aa, synthetic construct
  REGION 1..30: note = Synthetic Amino Acid Sequence
RVTITADTSTSTAYMELSSLRSEDTAVYYC

SEQ ID NO: 137 - protein, 11 aa, synthetic construct
  REGION 1..11: note = Synthetic Amino Acid Sequence
WGQGTLVTVSS

SEQ ID NO: 138 - protein, 25 aa, synthetic construct
  REGION 1..25: note = Synthetic Amino Acid Sequence
QVQLQESGPGLVKPSQTLSLTCTVS

SEQ ID NO: 139 - protein, 13 aa, synthetic construct
  REGION 1..13: note = Synthetic Amino Acid Sequence
WIRQPPGKGLEWI

SEQ ID NO: 140 - protein, 30 aa, synthetic construct
  REGION 1..30: note = Synthetic Amino Acid Sequence
RVTISVDTSKNQFSLKLSSVTAADTAVYYC

SEQ ID NO: 141 - protein, 11 aa, synthetic construct
  REGION 1..11: note = Synthetic Amino Acid Sequence
WGQGTLVTVSS

SEQ ID NO: 142 - protein, 11 aa, synthetic construct
  REGION 1..11: note = Synthetic Amino Acid Sequence
WGQGTLVTVSS

SEQ ID NO: 143 - protein, 15 aa, synthetic construct
  REGION 1..15: note = Synthetic Amino Acid Sequence
WYQQKPGKAPKLLIY

SEQ ID NO: 144 - protein, 32 aa, synthetic construct
  REGION 1..32: note = Synthetic Amino Acid Sequence
GVPSRFSGSGSGTDFTLTISSLQPEDFATYYC

SEQ ID NO: 145 - protein, 10 aa, synthetic construct
  REGION 1..10: note = Synthetic Amino Acid Sequence
FGQGTKVEIK

SEQ ID NO: 146 - protein, 23 aa, synthetic construct
  REGION 1..23: note = Synthetic Amino Acid Sequence
DIVMTQSPLSLPVTPGEPASISC

SEQ ID NO: 147 - protein, 15 aa, synthetic construct
  REGION 1..15: note = Synthetic Amino Acid Sequence
WYLQKPGQSPQLLIY

SEQ ID NO: 148 - protein, 32 aa, synthetic construct
  REGION 1..32: note = Synthetic Amino Acid Sequence
GVPDRFSGSGSGTDFTLKISRVEAEDVGVYYC

SEQ ID NO: 149 - protein, 10 aa, synthetic construct
  REGION 1..10: note = Synthetic Amino Acid Sequence
FGQGTKVEIK

SEQ ID NO: 150 - protein, 23 aa, synthetic construct
  REGION 1..23: note = Synthetic Amino Acid Sequence
DIVMTQSPDSLAVSLGERATINC

SEQ ID NO: 151 - protein, 15 aa, synthetic construct
  REGION 1..15: note = Synthetic Amino Acid Sequence
WYQQKPGQPPKLLIY

SEQ ID NO: 152 - protein, 32 aa, synthetic construct
  REGION 1..32: note = Synthetic Amino Acid Sequence
GVPDRFSGSGSGTDFTLTISSLQAEDFAVYYC

SEQ ID NO: 153 - protein, 10 aa, synthetic construct
  REGION 1..10: note = Synthetic Amino Acid Sequence
FGQGTKVEIK

SEQ ID NO: 154 - protein, 23 aa, synthetic construct
  REGION 1..23: note = Synthetic Amino Acid Sequence
DIVMTQSPDSLAVSLGERATINC

SEQ ID NO: 155 - protein, 15 aa, synthetic construct
  REGION 1..15: note = Synthetic Amino Acid Sequence
WYQQKPGQPPKLLIY

SEQ ID NO: 156 - protein, 32 aa, synthetic construct; identical to SEQ ID NO: 152
  REGION 1..32: note = Synthetic Amino Acid Sequence

SEQ ID NO: 157 - protein, 10 aa, synthetic construct
  REGION 1..10: note = Synthetic Amino Acid Sequence
FGQGTKVEIK

SEQ ID NO: 158 - protein, 673 aa, Homo sapiens
EPTMYGEILSPNYPQAYPSEVEKSWDIEVPEGYGIHLYFTHLDIELSENCAYDSVQIISGDTEEGRLCGQRSSNNPHSPIVEEFQVPYNKLQVIFKSDFSNEERFTGFAAYYVATDINECTDFVDVPCSHFCNNFIGGYFCSCPPEYFLHDDMKNCGVNCSGDVFTALIGEIASPNYPKPYPENSRCEYQIRLEKGFQVVVTLRREDFDVEAADSAGNCLDSLVFVAGDRQFGPYCGHGFPGPLNIETKSNALDIIFQTDLTGQKKGWKLRYHGDPMPCPKEDTPNSVWEPAKAKYVFRDVVQITCLDGFEVVEGRVGATSFYSTCQSNGKWSNSKLKCQPVDCGIPESIENGKVEDPESTLFGSVIRYTCEEPYYYMENGGGGEYHCAGNGSWVNEVLGPELPKCVPVCGVPREPFEEKQRIIGGSDADIKNFPWQVFFDNPWAGGALINEYWVLTAAHVVEGNREPTMYVGSTSVQTSRLAKSKMLTPEHVFIHPGWKLLEVPEGRTNFDNDIALVRLKDPVKMGPTVSPICLPGTSSDYNLMDGDLGLISGWGRTEKRDRAVRLKAARLPVAPLRKCKEVKVEKPTADAEAYVFTPNMICAGGEKGMDSCKGDSGGAFAVQDPNDKTKFYAAGLVSWGPQCGTYGLYTRVKNYVDWIMKTMQENSTPRED